(19) United States
(12) Patent Application Publication
(10) Pub. No.: US 2003/0203908 A1
(43) Pub. Date: Oct. 30, 2003

(54) Dihydrofolate reductase inhibitors

(76) Inventor: Gordon Lowe, Oxfordshire (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/182,047

(22) PCT Filed: Jan. 24, 2001
(86) PCT No.: PCT/GB01/00267

(30) Foreign Application Priority Data
Jan. 24, 2000 (GB) 0001565.1

(51) Int. Cl. C07D 251/48; A61K 31/53; A61K 31/277; A61K 31/155; C07C 279/26

(52) U.S. Cl. 514/245; 514/524; 514/635; 544/208; 564/234; 558/423

(57) ABSTRACT

A compound of formula (I) or (II), wherein X is hydrogen, halogen, alkyl, aralkyl,aryloxy, arylalkoxy or alkoxy, Y is hydrogen, halogen, alkyl, aralkyl, aryloxy, arylalkoxy or alkoxy, or formula (a) wherein A and B are different and one of A and B is CH₂ and the other is O, NH, or S or A and B are both CH₂ or CH═, and R^{a}, R^{b}, and R^{c} are the same or different and are hydrogen, halogen, alkyl, alkoxy, aryloxy, aralkyl or arylalkoxy. R¹ is hydrogen, and R² is hydrogen, C₁-C₆ alkyl or aryl which is unsubstituted or substituted by halogen, cyano, hydroxy, C₁-C₆ alkyl, which is unsubstituted or substituted by halogen, cyano, hydroxy, C₁-C₆ alkoxy, aralkyl, aryloxy or aryl, C₁-C₆ alkoxy, aralkyl, arylalkoxy, aryloxy or aryl, which is unsubstituted or substituted by halogen, cyano, hydroxy, C₁-C₆ alkyl or C₁-C₆ alkoxy; and pharmaceutically acceptable salts thereof; with the proviso that when R² is phenyl and Y is hydrogen, X is not chlorine.

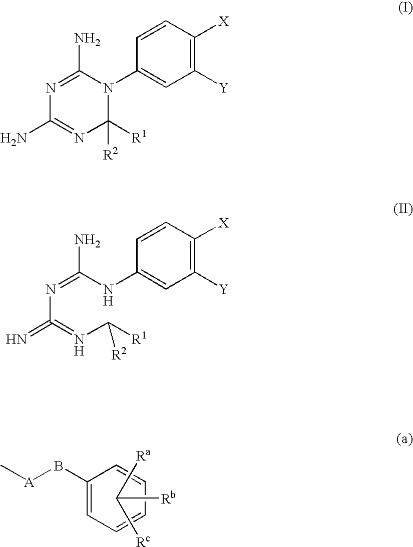

The present invention relates to novel dihydrofolate reductase inhibitors and their use as antiparasitic agents, for example against Plasmodium falciparum, Leishmania donovani, Trypanosoma cruzi and Trypanosoma brucei the causative agents of malaria, leishmaniasis, Chagas' disease (south American trypanosomiasis) and sleeping sickness (human African trypanosomiasis) respectively, Toxoplasma gondii which infects immunosuppressed patients including AIDS patients infected with HIV, and as antitumour agents.

Dihydrofolate reductase (DHFR) is an enzyme which catalyses the NADPH dependent reduction of dihydrofolate to tetrahydrofolate which is an essential cofactor in the biosynthesis of thymidine (as its mononucleotide), one of the four nuclear bases of DNA. Inhibition of DHFR leads to cell death due to the lack of thymidine for DNA synthesis.

Inhibition of DHFR makes it possible to treat a variety of diseases and infections as the relevant cells or infective agents are killed by cell death. However, there are considerable structural differences between DHFRs from human, bacterial and parasitic sources and this has led to the development of a variety of inhibitors. Most inhibitors are selective as anti-tumour, anti-bacterial or anti-parasitic agents. One parasitic agent which may be killed by DHFR inhibition is the malarial parasite Plasmodium falciparum. It is estimated there are 300-500 million clinical cases of malarial infections per year worldwide and that more than 2 million children die from the infection each year.

The dihydrofolate reductase (DHFR) domain of the Plasmodium falciparum bifunctional dihydrofolate reductase-thymidylate synthase (DHFR-TS) is an attractive target in malarial chemotherapy. Cycloguanil (A) and pyrimethamine (B) are potent inhibitors of the DHFR from Plasmodium falciparum (pf DHFR) and have been used extensively alone or in combination with other drugs as prophylactics and chemotherapeutic agents for the treatment of P.falciparum malaria. However, the rapid emergence of antifolate resistant P.falciparum has unfortunately compromised the clinical utility of these drugs, and there is an urgent need to find new effective antifolate antimalarials.

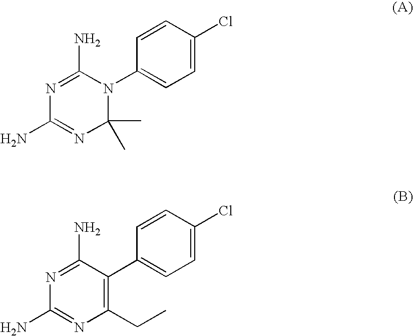

A variety of mutant P.falciparum parasites have evolved. Some parasites are resistant to one of (A) and (B) whereas others are resistant to both. Parasites with the mutations A16V and S108T are resistant to cycloguanil (A) but remain susceptible to pyrimethamine (B). Other sources of DHFR include, Leishmania major, Trypanosoma cruzi, Trypanosoma brucei, Leishmania donovani and Toxoplasma gondii.

Despite the considerable structural differences between DHFRs from human, bacterial and parasitic sources, some inhibitors have been developed which are able to inhibit DHFR from several sources.

Chagas' disease is caused by the protozoan parasite Trypanosoma cruzi. It is estimated that 16 to 18 million people are infected by this parasite and at present there are very few effective drugs for combatting this disease. Current drugs have poor clinical efficacy and give rise to severe side effects. Targeting DHFR as a target for therapeutic intervention in Chagas' disease is a new area.

WR99210 (C) was reported to be a potent inhibitor of plasmodium falciparum DHFR but it was never developed as a therapeutic agent because it showed severe gastro-intestinal side effects. However, it was subsequently recognised that the prodrug PS 15 (D) did not cause the gastro-intestinal problems associated with WR99210.

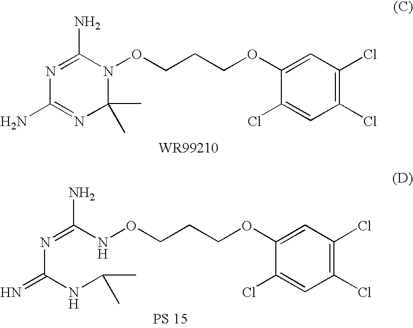

A novel class of compounds has now been found which are effective against these diseases and parasites.

The present invention provides novel dihydrofolate reductase inhibitors and compounds which may be metabolised to form dihydrofolate reductase inhibitors.

The present invention provides a pharmaceutical composition which comprises a compound of formula (I) or (II),

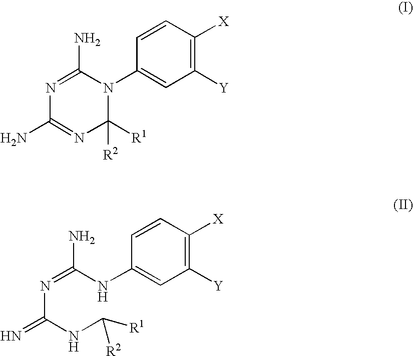

wherein X is hydrogen, halogen, alkyl, aralkyl, aryloxy, arylalkoxy or alkoxy,

Y is hydrogen, halogen, alkyl, aralkyl, aryloxy, arylalkoxy, alkoxy,

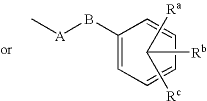

wherein A and B are different and one of A and B is CH₂ and the other is O, NH, or S or A and B are both CH₂ or CH═, and R^{a}, R^{b}, and R^{c} are the same or different and are hydrogen, halogen, alkyl, alkoxy, aryloxy, aralkyl or arylalkoxy.

R¹ is hydrogen, and

R² is hydrogen, C₁-C₆ alkyl or aryl which is unsubstituted or substituted by halogen, cyano, hydroxy, C₁-C₆ alkyl

which is unsubstituted or substituted by halogen, cyano, hydroxy, C₁-C₆ alkoxy, aralkyl, aryloxy or aryl,

C₁-C₆ alkoxy, aralkyl, aryloxy, arylalkoxy or aryl

which is unsubstituted or substituted by halogen, cyano, hydroxy, C₁-C₆ alkyl or C₁-C₆ alkoxy; and pharmaceutically acceptable salts thereof.

The compounds of formula (I) and (II) with the proviso that when R₂ is phenyl and Y is hydrogen, X is not chlorine, are believed to be novel and form another aspect of the present invention.

Where the compounds have isomers then all isomers, stereoisomers and enantiomers are included in the present invention.

X is preferably hydrogen, halogen, alkyl, aralkyl, or arylalkoxy, more preferably chlorine or benzyloxy.

Y is preferably hydrogen, halogen, alkyl, aralkyl, benzyloxy,

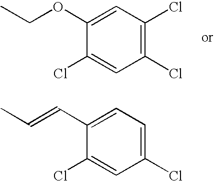

most preferably hydrogen, chlorine, benzyl or

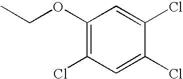

R² is preferably methyl or unsubstituted or substituted phenyl, more preferably phenyl substituted by phenoxy or benzyloxy.

Preferred compounds of the invention are compounds where X is chlorine or hydrogen and Y is hydrogen or chlorine.

Other preferred compounds are where R² is not hydrogen. Further preferred compounds are compounds where R² is substituted phenyl wherein the substituents are alkoxy, aryloxy or aryl.

In the present invention C₁-C₆ alkyl which may be straight or branched is preferably C₁-C₄ alkyl, for example methyl, ethyl, n-propyl, isopropyl, n-butyl, isobutyl, secbutyl, tertbutyl, more preferably methyl or ethyl, most preferably methyl. Halogen is preferably fluorine or chlorine more preferably chlorine. C₁-C₆ alkoxy may be straight or branched and is preferably C₁-C₄ alkoxy, for example methoxy, ethoxy, propoxy, n-propoxy, isopropoxy, n-butoxy, isobutoxy, sec-butoxy or tert-butoxy, more preferably methoxy or tert-butoxy most preferably methoxy.

Aryl, as used herein includes both monocyclic and bicyclic aromatic groups which typically contain from 6 to 10 carbons in the ring such as phenyl or naphthyl, preferably phenyl. Arylalkoxy is preferably benzyloxy. Aryloxy is preferably phenoxy.

In a preferred embodiment, the present invention provides compounds of formula (Ia).

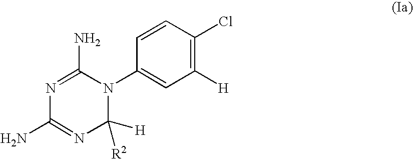

R² is phenyl substituted by alkyl, phenyl, alkoxy or benzyloxy or substituted phenyl, wherein the preferred substituents are methoxy, chlorine or tertbutyl, more preferably R² is 4-biphenyl, 4-ethoxyphenyl, 4-butyloxyphenyl, 4-benzyloxyphenyl, 3-benzyloxyphenyl, 4-phenoxyphenyl, 3-phenoxyphenyl, 3-(4-methoxyphenoxy)phenyl, 3-(4-chlorophenoxy)phenyl, 3-(4-t-butylphenoxy)phenyl or 4-isopropylphenyl more preferably 4-biphenyl, 4-butyloxyphenyl, 4-benzyloxyphenyl, 3-benzyloxyphenyl, 4-phenoxyphenyl, 3-phenoxyphenyl, 3-(4-methoxyphenoxy)phenyl or 3-(4-chlorophenoxy)phenyl.

In another preferred embodiment, the present invention provides compounds of formula (Ib)

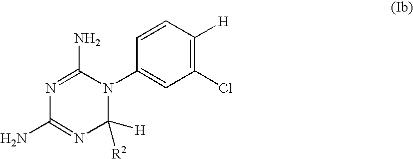

wherein R² is phenyl substituted by unsubstituted or substituted phenoxy or benzyloxy, wherein the preferred substituents are methoxy, chlorine, fluorine or tertbutyl, more preferably 4-phenoxyphenyl, 3-phenoxyphenyl, 3-(4-chlorophenoxy)phenyl, 3-(4-methoxyphenoxy)phenyl, 3-(4-t-butylphenoxy)phenyl, 3-benzyloxyphenyl, 4-(n-propyloxyl)phenyl, 3-(3,4-dichlorophenoxyl)phenyl, 4-(3,5-difluorobenzyloxy)phenyl or 3-(3,5-dichlorophenoxy)phenyl, more preferably phenoxyphenyl and most preferably 3-phenoxyphenyl.

In a further embodiment, the present invention provides compounds of formula (Ic)

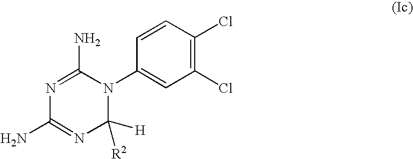

wherein R² is hydrogen or unsubstituted or substituted phenyl, preferably substituted phenyl especially by unsubstituted or substituted phenoxy such as methoxyphenoxy, most preferably 3-phenoxyphenyl or 3-(4-methoxyphenoxy)phenyl.

In a further embodiment, the present invention provides compounds of formula (Id).

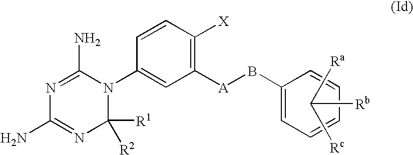

wherein X is hydrogen, halogen or alkyl, and R¹, R², A, B, R^{a}, R^{b} and R^{c} are as defined above. Preferably R^{a}, R^{b} and R^{c} are in the 2, 4 and 5 positions. Preferably A is CH₂, B is O and R^{a}, R^{b}, and R^{c} are all chlorine, and R^{a}, R^{b} and R^{c} are preferably in the 2, 4 and 5 positions, preferably R² is substituted phenyl, more preferably 3-benzyloxyphenyl. In another preferred embodiment of compounds of formula (Id) two of R^{a}, R^{b} and R^{c} are in the 2 and 4 positions. Preferably, A and B are both CH═ and R^{a} is hydrogen and R^{b} and R^{c} are both chlorine, preferably in the 2 and 4 positions, preferably R¹ is hydrogen and R² is substituted phenyl, more preferably R² is 3-phenoxyphenyl, 3-(4-chlorophenoxy)phenyl or 3-propyloxyphenyl.

In a further embodiment of the present invention R² is 3-benzyloxyphenyl and one of X or Y is benzyl or benzyloxy and the other is hydrogen. Another preferred embodiment of the present invention is when R² is 4-PrOC₆H₄, X is hydrogen and Y is benzyl or benzyloxy.

Preferred compounds of formula (II) correspond to the preferred compounds of formula (Ia), (Ib), (Ic) and (Id) above.

The compounds can be prepared by a one-pot process from the aniline, dicyandiamide and the carbonyl compound, or from the arylbiguanides with the aldehydes or from the aniline, dicyandiamide and dimethoxymethane.

Accordingly the present invention provides a process for the production of a compound of formula (III)

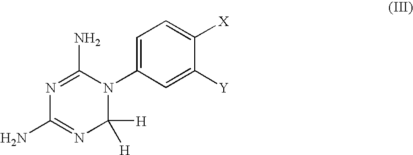

which process comprises reacting a compound of formula (IV)

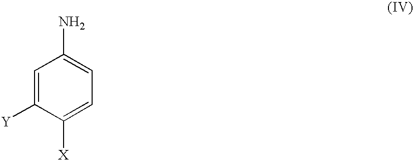

with dicyandiamide and dimethoxymethane, wherein X and Y are as defined above.

In another embodiment the present invention provides a process for the production of compounds of formula (V)

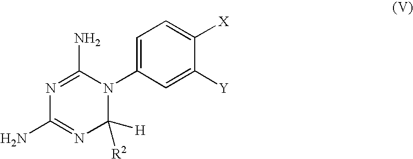

which process comprises reacting a compound of formula (VI)

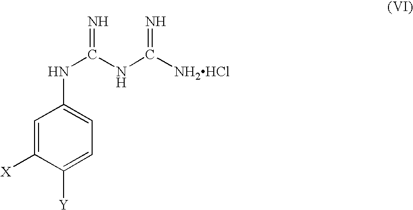

with a compound of formula (VII) or a ketal of (VII)

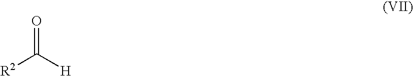

in the presence of a strong acid wherein X and Y are as defined above and

R² is C₁-C₆ alkyl or aryl which is unsubstituted or substituted by halogen, cyano, hydroxy, C₁-C₆ alkyl

which is unsubstituted or substituted by halogen, cyano, hydroxy, C₁-C₆ alkoxy, aralkyl, aryloxy or aryl,

C₁-C₆ alkoxy, aralkyl, aryloxy, aralkoxy or aryl

which is unsubstituted or substituted by halogen, cyano, hydroxy, C₁-C₆ alkyl or C₁-C₆ alkoxy; with the proviso that when R₂ is phenyl and Y is hydrogen, X is not chlorine.

A strong acid is an acid that is completely disassociated in aqueous solution, typically an acid with a pKa value of less than 1. Preferably the acid is hydrochloric acid, preferably concentrated hydrochloric acid, or p-toluene-sulphonic acid. Typically concentrated acid has a concentration of 10 molar.

Compounds of formula (II) can be prepared by a one-pot process from the aniline and dicyandiamide.

Accordingly the present invention provides a process for the production of a compounds of formula (VIII).

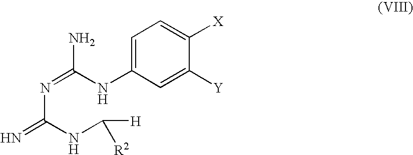

which process comprises reacting a compound of formula (IV)

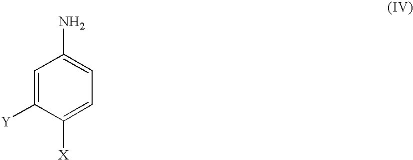

with a compound of formula (IX)

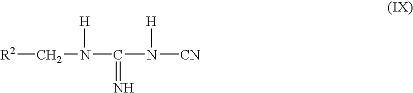

wherein X and Y

are as defined above

R² is aryl which is unsubstituted or substituted by halogen, cyano, hydroxy, C₁-C₆ alkyl

which is unsubstituted or substituted by halogen, cyano, hydroxy, C₁-C₆ alkoxy, aralkyl, aryloxy or aryl,

C₁-C₆ alkoxy, aralkyl, aryloxy, aralkoxy or aryl

which is unsubstituted or substituted by halogen, cyano, hydroxy, C₁-C₆ alkyl or C₁-C₆ alkoxy; with the proviso that when R² is phenyl and Y is hydrogen, X is not chlorine.

The process preferably takes place in a solvent which may be water.

The compounds used in the present invention may be used to inhibit dihydrofolate reductase (DHFR). The DHFR may be human, bacterial or parasitic. An example of parasitic DHFR is DHFR from Plasmodium falciparum.

The compounds may be used in the treatment or prophylaxis of diseases caused by parasites.

More specifically the compounds may be used to inhibit drug resistant Plasmodium falciparum, in particular DHFR from strains of Plasmodium falciparum which are resistant to cycloguanil such as T9/94.

The compounds may also be used in the treatment or prophylaxis of malaria, more particularly drug-resistant or multi-drug-resistant malaria, most preferably cycloguanil resistant malaria. Preferred compounds for use in treating malaria and in particular malaria caused by cycloguanil resistant P.falciparum are 1-p-chlorophenyl-2-p-isopropylphenyl-4,6-diamino-1,2-dihydro-1,3,5-triazine, 1-p-benzyloxyphenyl-2-p-n-butyloxyphenyl-4,6-diamino-1,2-dihydro-1,3,5-triazine, 1-p-benzyloxyphenyl-2-m-benzyloxyphenyl-4,6-diamino-1,2-dihydro-1,3,5-triazine, 1-p-chlorophenyl-2-p-n-butyloxyphenyl-4,6-diamino-1,2-dihydro-1,3,5-triazine and 1-m-benzylphenyl-2-m-benzyloxyphenyl-4,6-diamino-1,2-dihydro-1,3,5-triazine.

The compounds may also be used in the treatment or prophylaxis of Chagas' disease which is caused by the parasite Trypanosoma cruzi. Particularly preferred compounds for use in the treatment of Chagas' disease are 1-p-chlorophenyl-4,6-diamine-1,2-dihydro-1,3,5-triazine, 1-p-chlorophenyl-2-p-phenoxyphenyl-4,6-diamine-1,2-dihydro-1,3,5-triazine, 1-p-chlorophenyl-2-m-hydroxyphenyl-4,6-diamino-1,2-dihydro-1,3,5-triazine, 1-p-chlorophenyl-2-p-isopropylphenyl-4,6-diamine-1,2-dihydro-1,3,5-triazine, 1-p-chlorophenyl-2-p-ethoxyphenyl-4-6-diamino-1,2-dihydro-1,3,5-triazine, 1-p-chlorophenyl-2-p-methoxyphenyl-4,6-diamino-1,2-dihydro-1,3,5-triazine, 1-p-chlorophenyl-2-phenyl-4,6-diamino-1,2-dihydro-1,3,5-triazine, 1-[3-(2,4,5-trichlorophenoxymethyl)phenyl]-2,2-dimethyl-4,6-diamino-1,2-dihydro-1,3,5-triazine, 1-m-chlorophenyl-2-m-phenoxyphenyl-4,6-diamino-1,2-dihydro-1,3,5-triazine, 1-p-benzyloxyphenyl-2-m-phenoxyphenyl-4,6-diamino-1,2-dihydro-1,3,5-triazine and 1-p-chlorophenyl-2-m-phenoxyphenyl-4,6-diamino-1,2-dihydro-1,3,5-triazine, preferably 1-p-chlorophenyl-4,6-diamino-1,2-dihydro-1,3,5-triazine or 3-(2,4,5-trichlorophenoxymethyl)phenyl-2,2-dimethyl-4,6-diamino-1,2-dihydro-1,3,5-triazine, most preferably 1-p-chlorophenyl-4,6-diamino-1,2-dihydro-1,3,5-triazine.

The compounds may also be used in the treatment or prophylaxis of sleeping sickness and Toxoplasma gondii.

In a further embodiment, the compounds may be used in the manufacture of a medicament for inhibiting DHFR, preferably a medicament for treating malaria or Chagas' disease, more preferably a medicament for treating drug-resistant malaria, more preferably cycloguanil resistant or multidrug resistant malaria, most preferably inhibiting A16V-S108T pf DHFR.

The present compounds can be administered in a variety of dosage forms, for example orally such as in the form of tablets, capsules, sugar- or film-coated tablets, liquid solutions or suspensions or parenterally, for example intramuscularly, intravenously or subcutaneously. The present compounds may therefore be given by injection or infusion.

The dosage depends on a variety of factors including the age, weight and condition of the patient and the route of administration. Typically, however, the dosage adopted for each route of administration when a compound of the invention is administered alone to adult humans is in the range of 0.01 to 100 mg/kg body weight. Such a dosage may be given, for example, from 1 to 5 times daily by bolus infusion, infusion over several hours and/or repeated administration.

The DHFR inhibitors of formulae (I) and (II) or a pharmaceutically acceptable salt thereof are formulated for use as a pharmaceutical or veterinary composition also comprising a pharmaceutically or veterinarily acceptable carrier or diluent. The compositions are typically prepared following conventional methods and are administered in a pharmaceutically or veterinarily suitable form.

The present compounds may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example ethyl or n-propyl p-hydroxybenzoate, one or more colouring agents, and/or one or more sweetening agents such as sucrose, saccharin, glucose, sorbitol and mannitol.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occuring phosphatides, for example soy bean lecithin, and esters or partial esters derived from fatty acids and hexitol anhydrides, for example sorbitan mono-oleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents;

B) Parenterally, either subcutaneously, or intravenously, or intramuscularly, or intrasternally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols;

E) Topically, in the form of creams, ointments, jellies, collyriums, solutions or suspensions.

Daily dosages can vary within wide limits and will be adjusted to the individual requirements in each particular case. In general, for administration to adults, an appropriate daily dosage is in the range of about 5 mg to about 500 mg, although the upper limit may be exceeded if expedient. The daily dosage can be administered as a single dosage or in divided dosages.

### EXAMPLES

Preparation of Compounds

Compounds of the present invention were isolated as monohydrochloride salts and were purified by crystallisation from water or water-ethanol. New compounds gave clean ¹H nmr and mass spectra (APCI) and provide satisfactory elemental analysis (C,H,N).

Solvents were removed by rotary evaporation under reduced pressure using a Büchi Rotavapor R-114 equipped with a water bath and Vacuubrand CVC 2 vacuum pump. APCI mass spectra were recorded on a Fisons Instruments of VG Platform spectrometer equipped with an automated Hewlett Packard Series 1050 sample delivery system. Melting points were measured on a Reichert melting point apparatus and are uncorrected.

Thin layer chromatography was performed on aluminium sheets pre-coated with Merck silica gel 60 F₂₅₄. Spots were visualised under UV light. Routine ¹H 200 MHz NMR spectra were recorded on a Varian Gemini 200 FT spectrometer. 500 MHz spectra were recorded on a Bruker AMX 500 spectrometer. Chemical shifts are given in ppm referenced to the residual protonated solvent.

Methods and Materials

m-Chloroaniline was distilled under reduced pressure before use. All other chemicals were obtained from Sigma-Aldrich Ltd, Lancaster, Avocado Ltd, and BDH, and used as found. Reagent grade acetone and absolute alcohol were used.

Parallel synthesis reactions were performed in a Radley multiple synthesis block of 56 wells, equipped with a “Big Bill” rotating shaker, a water-cooled condensing stage, and a J-KEM programmable thermocouple and controller. Reagents were heated and refluxed in 4 ml capacity ReactiVials™ equipped with 10 cm condensing tubes inserted into teflon-coated seals and caps. Reactions involving highly volatile solvents were contained under SubaSeals vented using balloon leaks. Positive-ion chemical ionisation mass spectrometry was carried out as APCI in 1:1 methanol/dichloromethane as carrier solvent, or electrospray mass spectrometry in acetonitrile/formic acid. Elemental analysis (C, H, N) was performed at the Oxford University Inorganic Laboratory. 200 MHz NMR was carried out on a Varian Gemini 200, 250 MHz NMR on a Bruker AMX250, in commercially-obtained deuterated solvents.

Chemical Syntheses of 4,6-diamino-1,2-dihydro-1,3,5-triazine Analogues

Cycloguanil derivatives bearing gem-dimethyl groups at the C-2 position were prepared by a three-component condensation reaction between an aromatic amine, dicyanodiamide and acetone in the presence of concentrated aqueous HCl as described in the literature (Modest, E. J. Chemistry and Biological Studies on 1,2-Dihydro-s-triazines. II. Three-Component Synthesis. J. Org. Chem. 1956, 21, 1). When the carbonyl compound is an aldehyde, a two component condensation between the carbonyl compound and an aryl-biguanide, obtained from a reaction between an aromatic amine and dicyandiamide in the presence of HCl as catalyst (Modest, E. J.; Levine, P. Chemical and Biological Studies on 1,2-Dihydro-s-triazines. III. Two-component Synthesis. J.Org.Chem., 1956, 21, 14-20 and reference there cited) or a one pot reaction in which the biguanide was preformed, before addition of the carbonyl compound, was found to be superior. Derivatives of formaldehyde were prepared in the same way as other aldehydes, except for methylal was used as a source of formaldehyde. In most cases the desired products precipitated from the reaction medium (usually ethanol) as the crystalline hydrochloride salt. The samples for biological assays were recrystallized from ethanol or aqueous ethanol. All cycloguanil derivatives have been characterized by ¹H NMR, mass spectra (APCI) and elemental analysis (CHN).

Typical Procedure for the Synthesis of 4,6-diamino-1,2-dihydro-1,3,5-triazines by the Two Component Method

To a suspension of the arylbiguanide.HCl in an appropriate volume of absolute ethanol containing conc. HCl (0.5 eq) was added the aldehyde (1-2 eq). The reaction mixture was heated at reflux until the test for biguanide test was negative (30 min to several hours). On cooling in the fridge, a white crystalline solid precipitated which was collected by filtration and washed with ethanol, acetone then ether and air dried. Recrystallisation from water or ethanol-water give the pure sample.

The preparation of 3-aminobenzyl-2,4,5-trichlorophenylether and trans-1-(2,4-dichlorophenyl)-2-(3-nitrophenyl) ethylene was as described below.

3-Nitrobenzyl-2,4,5-trichlorophenylether

A suspension of 2,4,5-trichlorophenol (8.0 g, 40.5 mmol, 1.0 eq), 3-nitrobenzylchloride (7.0 g, 40.5 mmol, 1.0 eq), and KOH (2.24 g, 40.5 mmol, 1.0 eq) in diglyme:ethanol (1:1, 40 ml) was heated under reflux for 20 hours. The flocculent precipitate was collected, and washed in water, aqueous NaOH solution (0.1 M) and brine. The crude product was crystallised from methanol and to give the expected product as white crystals (7.2 g, 57%). Rf=0.73(100% CH₂Cl₂), m.p. 103-105° C. ¹H NMR (200 MHz, DMSO): δ 5.40 (s, 2H, CH₂); 7.61 (s, 1H, ClCCHCCl); 7.71 (t, J 8.5 Hz, 1H, H_{b}); 7.86 (s, 1H CICCHCO); 7.90 (d, J 8.5 Hz, 1H, Hₐ); 8.21 (d, J 8.5 Hz, 1H, H_{c}); 8.31 (s, 1H, H_{d}), m/z 332, 334, 336 [M+H]⁺

3-Aminobenzyl-2,4,5-trichlorophenylether

A mixture of 3-nitrobenzyl-2,4,5-trichlorophenylether (0.5 g, 1.5 mmol, 1.0 eq.) and SnCl₂. 2H₂O in ethanol (70 ml) was stirred and heated under reflux for 12 hours. The mixture was diluted with a saturated solution of aqueous NaHCO₃. The aqueous phase was extracted with ethyl acetate. The combined organic phases were stirred for 1 hour with saturated aqueous KF to destroy the tin complex and then dried over Na₂SO₄. The solvent was removed under reduced pressure to yield the expected product as white solid (455 mg, 100%). R_{f}=0.36 (100% CH₂Cl₂),.m.p. 83-85° C. ¹H NMR (200 MHz, CDCl₃): δ 3.75 (s, 2H, NH₂); 5.06 (s, 2H, CH₂); 6.67 (dd, J 1.32 Hz, 8.0 Hz, 1H, Hₐ); 6.78 (s, 1H, H_{d}); 6.81 (d, J 8.0 Hz,1H, H_{c}); 7.05 (s, 1H, ClCCHCCl); 7.22 (t, J 8.0 Hz, 1H, Hb; 7.48 (s, 1H, ClCCHCO). m/z 302, 304, 306 [M+H]⁺.

3-Nitrobenzyltriphenylphosphonium chloride

A solution of 3-nitrobenzylchloride (3.0 g, 17 mmol, 1.0 eq) in dry benzene (20 ml) was added dropwise to a solution of triphenylphosphine (4.58 g, 17 mmol, 1.0 eq) in dry benzene (20 ml). The mixture was stirred and heated under reflux for 48 hours, then cooled to room temperature and filtered to give the product as pale brown crystals (4.93 g, 65%). ¹H NMR (200 MHz, CDCl₃): δ 6.00 (d, J_{PH} 16 Hz, 2H, CH₂); 7.90-8.20 (m,19H, Hₐᵣ), m/z 399 [M+H+Cl]⁺.

1-(2,4-Dichlorophenyl)-2-(3-nitrophenyl)ethylene

3-Nitrobenzyltriphenylphosphonium chloride (1.3 g, 3.0 mmol, 1.05 eq.), and 2,4-dichlorobenzaldehyde (0.5 g, 2.86 mmol, 1 eq.) were dissolved in CH₂Cl₂ (3.6 ml). The mixture was stirred as vigorously as possible, and aqueous NaOH (50%, 1.56 ml) was added dropwise. The solution became dark. The mixture was stirred for a further 30 minutes at room temperature. The aqueous phase was extracted with CH₂Cl₂. The organic phases were combined and dried over Na₂SO₄. The solvent was removed under reduced pressure to yield a crude solid. Purification by chromatography on silica (1:1 hexane:CH₂Cl₂) gave the product as yellow crystals (747 mg, 89%). Rf-0.7 (100% CH₂Cl₂). Mixture of two geometric isomers (83:17 cis:trans). Only the major product is described in ¹H NMR data.

¹H NMR (500 MHz, CDCl₃): 6.83 (ABX, 2H, H₁, H₂); 6.08 (d, J₇₋₈ 8.0 Hz, 1H, H₇); 6.11 (dd, J₈₋₇ 8.0 Hz, J₈₋₉ 2.0 Hz, 1H, H₈); 7.41 (t, J₄₋₃=J₄₋₅ 8.0 Hz, 1H, H₄); 7.47 (d, J₃₋₄8.0 Hz, 1H, H₃); 7.52 (d, J₉₋₈ 2.0 Hz, 1H, H₉); 7.08 (s, 1H, H₆); 8.10 (d, J₅₋₄ 1.0 Hz, 1H, H₅).

Mass spectrum: m/z 291; 294 [M−H]⁻

trans-1-(2,4-Dichlorophenyl)-2-(3-nitrophenyl)ethylene

1-(2,4-Dichlorophenyl)-2-(3-nitrophenyl)ethylene (mixture of stereoisomers) (3.0 g, 11.36 mmol) was dissolved in nitrobenzene (12 ml). A few crystals of iodine were added. The mixture was stirred and heated under reflux for 2 hours, then cooled at room temperature. The mixture was diluted with ethyl acetate, and washed with saturated aqueous sodium thiosulphate. The solvent was removed under reduced pressure and the crude mixture obtained was crystallised from AcOEt/hexane to give the product as yellow crystals (2.4 g, 78%). Rf=0.7 (100% CH₂Cl₂). m.p. 144-146° C. ¹H NMR (500 MHz, CDCl₃)_{:}δ 7.15 (d, J₂₋₁ 16.3 Hz, 1H, H₂); 7.34 (dd, J₈₋₇ 8.5 Hz, J₈₋₉ 2 Hz, 1H, H₈); 7.50 (d, J₉₋₈ 2.0 Hz, 1H, H₉); 7.61 (d, J₁₋₂ 16-3 Hz, 1H, H,); 7.61 (t, J₄₋₃=J₄₋₃ 7.9 Hz, 1H, H₄); 7.68 (d J₇₋₈ 8.5 HZ, 1H, H₇); 7.90 (d, J₃₋₄ 7.8 Hz, 1H, H₃); 8.20 (dd, J₅₋₄ 8.0 Hz, J₅₋₆ 1.9 Hz, 1H, H₅); 8.43 (t, J₆₋₅=J₆₋₃ 1.9 Hz, 1H, H₆). ¹³C NMR (500 MHz, CDCl₃): δ121.43 (s, 1C, C═): 122.78 (s, 1C, C═); 126.66 (s, 1C, Car); 127.36 (s, 1C, Car); 127.49 (s, 1C, Car); 129.15 (s, 1C, Car); 129.70 (s, 1C, Car); 129.76 (s, 1C, Car); 132.39 (s, 1C, Car); 133.04 (s, 1C, C-C═); 134.27 (s, 1C, C—Cl); 134.46 (s, 1C, C—Cl); 138.51 (s, 1C, C—C═); 148.71 (s, 1C, C—NO₂).

Trans-1-(2,4-Dichlorophenyl)-2-(3-aminophenyl)ethylene

A mixture of 1-(2,4-dichlorophenyl)-2-(3-nitrophenyl)ethylene (4.9 g, 0.02 mmol, 1.0 eq.) And SnCl₂. 2H₂O (22.6 g, 0.1 mol, 5.0 eq.) in ethanol (700 ml) were stirred and heated under reflux for 12 hours. The mixture was diluted with saturated aqueous NaHCO₃. The aqueous phase was extracted with ethyl acetate. The organic phases were combined, and stirred for 1 hour with saturated aqueous KF to destroy the tin complex. The organic phases were dried over Na₂SO₄. The solvent was removed under reduced pressure and to give the product as yellow crystal (5.13 g, 97%). R_{f}=0.35 (100% CH₂Cl₂).

¹H NMR (500 MHz, CDCl₃): δ 3.73 (s, 2H, NH₂); 6.88 (t, J₆₋₃=J₆₋₅ 1.9 Hz, 1H, H₆); 6.95 (d, J₅₋₄ 7.8 Hz, 1H, H₅); 6.98 (d, J₂₋₁ 16.5 Hz, 1H, H₂); 7.18 (t,J₄₋₃=J₄₋₅ 7.8 Hz, 1H, H₄); 7.24 (dd, J₈₋₇ 8.6 Hz, J₈₋₉ 2.3 Hz, 1H, H₈); 7.38 (d, J₁₋₂ 16.5 Hz, 1H, H₁); 7.41 (d, J₉₋₈ 2.2 Hz, 1H, H₉); 7.60 (d, J₇₋₈ 8.5 Hz, 1H, H₇).

¹³C NMR (500 MHz, CDCl₃): δ 112.93 (s, 1C, Car); 115.30 (s,1C, Car); 117.78 (s, 1C, Car); 123.41 (s, 1C, C═); 127.13 (s, 1C, Car); 127.26 (s, 1C, Car); 129.51 (s, 1C, Car,); 129.67 (s, 1C, Car); 131.88 (s, 1C, C═); 133.26 (s, 1C, C_{q}); 133.77 (s, 1C, C_{q}); 134.08 (s, 1C, C—C1); 137.78 (s, 1C, C—C1); 146.69 (s,1C, C—NH₂). m/z 264; 266; 268 [M+H]+

Analytical data for some compounds of formula (I) is given below.

1-(4′-Chlorophenyl)-4,6-diamino-1,2-dihydro-1,3,5-triazine hydrochloride

δ¹H (DMSO-d₆) 4.71 (2H, s, CH₂), 6.98 (1H, s br ex., NH), 7.48 (4H, dd, J_{AB}=8, Ar—C—H), 7.65 (2H, s, NH₂), 7.85 (1H, s br ex., NH), 8.74 (1H, s, NH⁺); m/z 224 (MH+).

1-(phenyl)-4,6-diamino-1,2-dihydro-1,3,5-triazine hydrochloride

δ¹H (DMSO-d₆) 4.78 (2H, s, CH₂), 6.89 (1H, s br ex., NH), 7.34-7.58 (m, 7H, Ar—C—H and NH₂), 7.80 (1H, s br ex., NH) 8.58 (1H, s, NH⁺); m/z 190 (MH+).

1-(4′-Fluorophenyl)-4,6-diamino-1,2-dihydro-1,3,5-triazine hydrochloride

δ¹H (DMSO-d₆) 1.31 (6H, s, 2×Me), 6.42 (1H, s br ex., NH), 7.29-7.47 (6H, m, Ar—C—H and NH₂), 7.67 (1H, s br ex., NH), 9.01 (1H, s, NH⁺). δ¹³C (DMSO-d₆) 27.6 (2C, 2×Me), 70.1 (1C, CMe₂), 117.4+117.9 (2C, Ar—C—F ortho), 131.7 (1C, CN₃), 133.0+133.2 (2C, Ar—C—F meta), 158.24 (1C, Ar—C—F ipso), 160.1 (1C, CN₃), 165.6 (1C, Ar—C—N). ¹⁹F (DMSO-d₆, 250 MHz)−112.5 (1F, Ar—F); m/z 208 (MH+).

1-(4′-Chlorophenyl)-2-phenyl-4,6-diamino-1,2-dihydro-1,3,5-triazine hydrochloride

δH(D₂O, 200 MHz) 5.84 (1H, s, H-2), 6.90 (2H, part of AB doublet, J=8 Hz, aromatic C—H), 7.05 (7H, m, aromatic C—H); m/z 300 (100%, M.H+).

1-(3′-Chlorophenyl)-2-phenyl-4,6-diamino-1,2-dihydro-1,3,5-triazine hydrochloride

δH (D₂O, 200 MHz) 5.95 (1H, s, H-2), 6.90-7.30 (m, 9H, aromatic C—H); m/z 300 (100%, M.H+).

1-(3′,4′-Dichlorophenyl)-2-phenyl-4,6-diamino-1,2-dihydro-1,3,5-triazine hydrochloride

δH (D₂O, 200 MHz) 5.92 (1H, s, H-2), 6.90 (1H, dd J=8.0, 2.5 Hz, aromatic-CH), 7.10-7.30 (7H, m, aromatic CH); m/z 334 (100%, M.H+).

1-p-Benzyloxyphenyl-2-m-phenoxyphenyl-4,6-diamino-1,2-dihydro-1,3,5-triazine

¹H NMR (400 MHz, CD₃OD): 5.07 (s, 2H, CH₂O); 5.88 (s, H, CH); 6.84 (d, J 8.7 Hz, 2H, H₂, H₆); 7.01 (m, 4H); 7.13 (d, J 8.7 Hz, H₃, H₅); 7.38 (m, 10H). m/z 464 [M+H]⁺. m.p. 208-210° C. Calculated N, 13.5; C, 65.0; H, 5.4 (M+H₂O).Found N, 13.6; C, 65.0; H, 5.3%.

1-p-benzyloxyphenyl-2-p-n-butoxyphenyl-4,6-diamino-1,2-dihydro-1,3,5-triazine

¹H NMR (400 MHz, CD₃OD): 0.98 (t, J 7.3 Hz, 3H, CH₃); 1.49 (s^{t}, J 7.3 Hz, 2H, CH₂); 1.73 (q^{t}, J 7.2 Hz, 2H, CH₂); 3.96 (t, J 7.3 Hz, 2H, CH₂O); 5.06 (s, 2H, CH₂O(Bn)); 5.85 (s, H, CH); 6.88 (d, J 8.7 Hz, 2H, H₂, H₆); 6.99 (m, 5H); 7.22 (d, J 8.7 Hz, H₃, H₅); 7.38 (m, 4H).

m/z 444 [M+H]⁺.m.p.185-186° C. Calculated N, 13.6; C, 63.0; H, 7.1; (M+1EtOH), Found N, 13.8; C, 62.9; H, 7.5%.

1-p-benzyloxyphenyl-2p-n-propyloxyphenyl-4,6-diamino-1,2-dihydro-1,3,5-triazine

Crystallized from THF and small amount of methanol.

¹H NMR (400 MHz, CD₃OD): 1.07 (t, J 7.4 Hz, 3H, CH₃); 1.82 (m, 2H, CH₂); 3.95 (t, J 6.4 Hz, 2H, CH₂O); 5.10 (s, 2H, CH₂O(Bn)); 5.89 (s, H, CH); 6.92 (d, J 8.7 Hz, 2H, H_{3″}, H_{5″}); 7.04 (m, 4H, H₂, H₃, H₅, H₆); 7.26 (d, J 8.7 Hz, 2H, H_{2″}, H_{6″}); 7.35 (q, J 7.1 Hz, 1H, H_{4′}); &0.4 (t, J 7.1 Hz, 2H, H_{3′}, H_{5′}); 7.44 (d, J 7.1 Hz, 2H, H_{2′}, H_{6′}).

m/z 430 [M+H]⁺ m.p. 148-150° C. Calculated N, 13.0; C, 65.7; H, 6.7; (M+THF). Found N, 13.0; C, 66.1; H, 6.2%.

1-p-benzyloxyphenyl-2-m-benzyloxyphenyl-4,6-diamino-1,2-dihydro-1,3,5-triazine

¹H NMR (400 MHz, CD₃OD): 5.08 (s, 2H, CH₂O(Bn)); 5.10 (s, 2H, CH₂O(Bn)); 5.86 (s, H, CH); aromatic protons (18H).

m/z 477 [M+H]⁺.m.p. 187-189° C. Calculated N, 13.6; C, 67.7; H, 5.5; Found N, 13.6; C, 67.6; H, 5.5%.

1-m-biphenyl-2-m-benzyloxyphenyl-4,6-diamino-1,2-dihydro-1,3,5-triazine

¹H NMR (400 MHz, CD₃OD):5.08 (s, 2H, CH₂O); 6.00 (s, H, CH); 6.95 (d, J 7.7 Hz, 1H, H_{6″}); 7.01 (t, J 1.9 Hz, 1H, H_{2″}); 7.06 (dd, J 8.5 Hz, J 1.9 Hz, 1H, H_{4″}); 7.07 (d, J 7.1 Hz, 1H, H₆); 7.27 (broad s, 1H, H₂); 7.33 (m, 4H, H_{3′″}, H_{4′″}, H_{5′″}, H_{5″}); 7.37 (d, J 7.5 Hz, 2H, H_{2′″}, H_{6′″}); 7.38 (t, J 7.1 Hz, 1H, H_{4′}); 7.43 (t, J 7.1 Hz, 2H, H_{3′}, H_{5′}); 7.47 (t, J 7.7 Hz, 1H, H₅); 7.49 (d, J 7.1 Hz, 2H, H_{2′}, H_{6′}); 7.66 (d, J 7.9 Hz, 1H, H₄). m/z 448 [M+H]⁺, m.p. 169-71° C. Calculated N, 14.4; C, 69.4; H, 5.4; Found N, 14.2; C, 68.9; H, 5.6%.

1-m-biphenyl-2-m-phenoxyphenyl-4,6-diamino-1,2-dihydro-1,3,5-triazine

¹H NMR (400 MHz, CD₃OD): 6.06 (s, H, CH); 6.81 (d, J 7.7 Hz, 2H, H_{2′″}, H_{6′″}); 6.92 (t, J 2.1 Hz, 1H, H₂); 7.02 (dd, J 8.1 Hz, 2.3 Hz, 1H, H₄); 7.10 (tt, J 7.5 Hz, 1.1 Hz, 1H, H_{4″}′); 7.17 (d, J 7.7 Hz, 1H, H_{6″}); 7.22 (d, J 7.8 Hz, 1H, H₆); 7.26 (t, J 7.5 Hz, 2H, H_{5′″}, H_{3′″}); 7.34 (s, 1H, H_{2″}); 7.42 (m, 2H, H₅, H_{4′}); 7.46 (t J 7.1 Hz, 2H, H_{3′}, H_{5′}); 7.52 (d, J 7.0 Hz, 2H, H_{2′}, H_{6′}); 7.53 (t, J 7.8 Hz, 1H, H_{5″}); 7.71 (d, J 7.8 Hz, 1H, H_{4″}). m/z 434 [M+H]⁺, m.p. 165-168° C. Calculated N, 13.8; C, 64.9; H, 5.6; (M+2H₂O).

Found N, 13.94; C, 65.3; H, 5.7%.

1-m-benzyloxyphenyl-2-m-benzyloxyphenyl-4,6-diamino-1,2-dihydro-1,3,5-triazine

¹H NMR (400 MHz, CD₃OD): 4.98 (ABX, J 6.4 Hz, 1.9 Hz, 2H, CH₂O(Bn)); 5.09 (s, 2H, CH₂O(Bn)); 5.91 (s, H, CH); 6.66 (d, J 7.6 Hz, 1H, H₆); 6.72 (t, J 2.1 Hz, 1H, H₂); 6.88 (d, J 7.5 Hz, 1H, H₄); 6.97 (s, 1H, H_{2′}); 7.02 (m, 2H); 7.30-7.60 (m, 12H).

m/z 478 [M+H]⁺.m.p. 190-192° C. Calculated N, 13.6; C, 63.3; H, 5.3; Found N, 14.1; C, 63.6; H, 5.4%.

1-m-benzylphenyl-2-m-benzyloxyphenyl-4,6-diamino-1,2-dihydro-1,3,5-triazine

¹H NMR (400 MHz, CD₃OD): 3.98 (s, 1H, CH₂); 5.14 (s, 2H, CH₂O); 5.77 (s, H, CH); 7.05 (d, J 7.7 Hz, 1H, H_{6″}); 7.10 (d, J 2.1 Hz, 1H, H_{2″}); 7.12 (m, 2H, H₄, H_{4″}); 7.16 (broad s, 1H, H₂); 7.19 (m, 1H, H_{4′}); 7.20 (d, J 7.0 Hz, 2H, H_{2′}, H_{6′}); 7.26 (m, 3H, H_{3′}, H_{5′} H₆);.7.31 (m, 2H, H_{4′″}, H₅); 7.37 (t, J 7.1 Hz, 2H, H_{3′″}, H_{5′″}); 7.40 (t, J 7.8Hz, 1H, H_{5″}); 7.44 (d, J 7.3 Hz, 2H, H_{2′″}, H_{6′″}). m/z 462 [M+H]⁺. m.p. 165-167° C.

Calculated N, 13.5; C, 67.5; H, 5.9; (M+H₂O):Found N, 13.5; C, 67.1; H, 5.9%.

1-m-benzylphenyl-2-p-n-butyloxyphenyl-4,6-diamino-1,2-dihydro-1,3,5-triazine

¹H NMR (400 MHz, CD₃OD): 0.98 (t, J 7.5 Hz, 3H, CH₃); 1.51 (s^{t}, J 7.6 Hz, 2H, CH₂); 1.75 (q^{t}, J 6.4 Hz, 2H, CH₂); 3.88 (s, 2H, CH₂); 3.93 (t, J 6.4 Hz, 2H, CH₂O); 4.85 (s, 1H, CH); 6.81 (d, J 8.7 Hz, 2H, H_{3″}, H_{5″}); 6.86 (broad s, 1H, H₂); 7.00 (d, J 7.9 Hz, 2H, H_{2′}, H_{6′}); 7.06 (m, 1H, H₆);.7.15 (d, J 8.8 Hz, 2H, H_{2″}, H_{6″}); 7.17-7.24 (m, 4H, H_{3′}, H_{4′}, H_{5′}, H₄); 7.31 (t, J 7.8 Hz, 1H, H₅). m/z 428 [M+H]⁺. m.p. 130-132° C. Calculated N, 15.1%, C, 67.3; H, 6.5; Found N, 15.1; C, 66.6; H, 6.6%.

1-m-benzylphenyl-2-p-n-propyloxyphenyl-4,6-diamino-1,2-dihydro-1,3,5-triazine

¹H NMR (400 MHz, CD₃OD): 1.06 (t, J 7.5 Hz, 3H, CH₃); 1.82 (s^{t}, J 7.6 Hz, 2H, CH₂); 1.75 (q^{t}, J 6.4 Hz, 2H, CH₂); 3.90 (t, J 6.4 Hz, 2H, CH₂O); 3.92 (s, 2H, CH₂); 5.95 (s, 1H, CH); 6.83 (d, J 8.7 Hz, 2H, H_{3″}, H_{5″}); 6.9 (broad s, 1H, H₂); 7.03 (d, J 7.4 Hz, 2H, H_{2′}, H_{6′}); 7.05 (m, 1H, H₆);.7.17 (d, J 8.8 Hz, 2H, H_{2″}, H_{6″}), 7.19 (t, J 6.9 Hz, 1H, H_{4′}); 7.23-7.27 (m, 3H, H_{3′}, H_{5′}, H₄); 7.33 (t, J 7.8 Hz, 1H, H₅). m/z 414 [M+H]⁺. m.p. 142-143C. Calculated N, 14.9; C, 64.2; H, 6.5;(M+H₂O): Found N, 14.7; C, 64.8; H, 6.6%.

1-m-benzylphenyl-2-m-phenoxyphenyl-4,6-diamino-1,2-dihydro-1,3,5-triazine

¹H NMR (400 MHz, CD₃OD): 3.94 (s, 1H, CH₂); 5.94 (s, H, CH); 6.82 (d, J 7.7 Hz, 2H, H_{2′}, H_{6′}); 6.84 (t, J 2.0 Hz, 1H, H_{2″}); 6.93 (broad s, 1H, H₂); 7.07 (ddd, J 8.2 Hz, J 2.1 Hz, J 0.9 Hz, 1H, H_{4″}); 7.03 (broad d, J 7.9 Hz, 1H, H₆); 7.07 (d, J 7.1 Hz, 2H, H_{2′″}, H_{6′″}); 7.09 (d, J 8.2 Hz, 1H, H_{6′});.7.14 (tt, J 7.4 Hz, J 1.1 Hz, 1H, H_{4′}); 7.19 (t, J 7.4 Hz, 1H, H_{4′″}); 7.26 (tt, J 7.5 Hz, J 1.5 Hz, 1H, H_{3′″}, H_{5′″}); 7.30 (broad d, J 7.8 Hz, 1H, H₄); 7.32-7.36 (m,3H, H_{3′}, H_{5′}, H_{5″}); .7.38 (t, J 7.8 Hz, 1H, H₅). m/z 448 [M+H]⁺. m.p. 165-167° C. Calculated N, 14.0; C, 66.9; H, 6.6; (M+H₂O): Found N, 14.6; C, 66.8; H, 6.4%.

1-[3′-(2,4,5-trichlorophenoxy)-n-propyloxy]-2-p-n-propyloxyphenyl-4,6-diamino-1,2-dihydro-1,3,5-triazine

¹H NMR (200 MHz, CD₃OD): 1.00 (t, J 7.4 Hz, 3H, CH₃); 1.74 (s^{t}, J 7.4 Hz, 2H, CH₂ₑ); 2.00 (m, 2H, CH_{2b}); 3.78 (m, 5H, 2H_{b}, 2H_{c}, 1Hₐ); 4.10 (dt, J 9.5 Hz, J 6.8 Hz, 1H, H_{a′}); 5.72 (s, 1H, CH); 6.85 (d, J 8.7 Hz, 2H, H_{2′}, H_{6′}); 7.08 (s, 1H, H₃); 7.36 (d, J 8.7 Hz, 2H, H_{3′}, H_{5″}); 7.56 (s, 1H, H₆).

m/z 500, 502, 504, 506 [M+H]⁺. m.p. 198-200° C. Calculated N, 12.6; C, 45.5; H, 4.7; (M+H₂O): Found N, 12.5; C, 46.2; H, 4.8%.

1-[3′-(2,4,5-trichlorophenoxy)-n-propyloxy]-2-p-n-butyloxyphenyl-4,6-diamino-1,2-dihydro-1,3,5-triazine

¹H NMR (400 MHz, CD₃OD): 0.97 (t, J 7.4 Hz, 3H, CH₃); 1.46 (s^{t}, J 7.4 Hz, 2H, CH_{2f}); 1.71 (q^{t}, J 7.3 Hz, 2H, CH₂ₑ); 2.00 (m, 2H, CH_{2b}); 3.7-3.9 (m, 5H, 2H_{b}, 2H_{c}, 1Hₐ); 4.10 (dt, J 12.3 Hz, J 6.5 Hz, 1H, H_{a′}); 5.73 (s, 1H, CH); 6.82 (d, J 8.7 Hz, 2H, H_{2′}, H_{6′}); 7.08 (s, 1H, H₃); 7.35 (d, J 8.7 Hz, 2H, H_{3′}, H_{5′}); 7.56 (s, 1H, H₆). m/z 514, 516, 518, 520 [M+H]⁺. m.p. 190-192° C. Calculated N, 12.3; C, 44.9; H, 4.8, (M+1HCl):Found N, 13.0; C, 44.6;H, 5.4%.

General Procedure for the Preparation of Compounds of Formula (II)

Benzylamine hydrochloride or a substituted-benzylamine hydrochloride (0.1 mole), sodium dicyanamide (0.1 mole) in butanol (50 ml) was refluxed with stirring for 3 h. The cooled suspension was filtered and the filtrate evaporated. The residual syrup solidified on trituration (usually with dioxane) and the (substituted)-benzyldicyandiamide recrystallised (usually from dioxane). Substituted-aniline hydrochloride (0.1 mole), benzyldicyandiamide or substituted benzyldicyandiamide (0.1 mole) and water (50 ml) were refluxed for 4 h. The cooled suspension was filtered and the colourless crystals recrystallised generally from aqueous ethanol to give N¹-(substituted)-phenyl-N⁵-(substituted)-benzylbiguanide hydrochloride.

Preparation of P.falciparum DHFR and Inhibitor Binding Studies

The recombinant plastids pET-pfDHFR (wild-type) and pET-pfDHFR (A16V+S108T) were prepared as described in the literature (Sirawaraporn, W.; Sathitkul, T.; Sirawaraporn, R.; Yuthavong, Y.; Santi, D. V. Antifolate-resistant mutants of Plasmodium falciparum dihydrofolate reductase. Proc. Natl. Acad. Sci. USA, 1997, 94 1124-29) and were employed for the preparation of enzymes used for testing the compounds of the present invention. Purification of both wild-type and mutant pfDHFRs was achieved by affinity chromatography on MTX-Sepharose CL-6B column (Sirawaraporn, W.; Prapunwattana, P.; Sirawaraporn, R.; Yuthavong, Y.; Santi, D. V. The Dihydrofolate Reductase Domain of Plasmodium falciparum Thymidylate Synthase-Dihydrofolate Reductase. J. Biol Chem. 1993, 29, 2163-744). The activity of pfDHFR was determined spectrophotometrically by monitoring the decrease in absorbance at 340 nm at 25° C. according to the standard assay method in the literature (J. Biol Chem. 1993, 29, 21637-44). For determination of the Ki values of a number of synthesized analogues, the assay reactions were initiated with affinity-purified enzyme (^{˜}0.005-0.01 U ml⁻¹), and the initial velocities in the presence of varying concentrations of inhibitors were analysed by non-linear least square fit using Kaleidagraph™ software. Other analyses of the protein were performed as described in the literature.

Folic acid and NADPH, were purchased from Sigma. Dihydrofolate was prepared from folic acid dithionate reduction. Methotrexate (MTX) was from Lederle Laboratories. MTX sepharose CL-6B was prepared according to the protocol described by Dann. et al. IPTG came from Boehringer Manheim. The expression system used for recombinant human DHFR production, which includes the plasmid PB-E1/RB SII, SphI-HDHFR expressed in E. coli M15, was supplied by Santi, D. V (Department of Biochemistry and Biophysics and Department of Pharmaceutical Chemistry, University of California). Recombinant T. Cruzi DHFR-TS was produced using the system pET 11c with the insertion of the complete ORF of the gene tc-dhfrts. The resulting plasmid was called pET DT.

Human and T.cruzi DHFR were produced using inducible expression systems. For human DHFR, fresh overnight cultures of E. coli M15, transformed with the plasmid, pB-E1/RB SII, SphI-HDHFR, were grown in LB and 0.1 mg/ml ampicillin medium at 28° C. For T.cruzi DHFR-TS, cultures of E. coli, BL21 (DE3) transformed with the plasmid pETDT system were grown at 37° C. The same purification protocol was used for both systems. When OD600 was at 0.8, cells pellets were collected by centrifugation, suspended in 50 ml of LB medium, containing 0.1 mg/ml of ampicillin. 10 ml of this preinocule was added to 2 litres of LB medium. Cultures were shaken at 28° C. or 37° C. (for human and T. cruzi DHFR respectively) and induction with IPTG ImM was performed when OD600 reached 0.8. After IPTG induction, cells were grown overnight in a shaker at 28° C. or 5 hours at 28° C. or 37° C. (for human and T. cruzi DHFR respectively) and then harvested by centrifugation.

Cell pellets were suspended in sonication buffer (2× TES DHFR:TES 100 mM, 2-mercaptoethanol 150 mM, EDTA 2 mM pH=7) containing protease inhibitors. Crude extracts were obtained by sonication and centrifugation and loaded onto a 5 ml MTX sepharose affinity chromatography CL-6B column, pre-equilibrated with buffer KH₂PO₄ 10 mM, pH 7.

Crude extract was circulated through the MTX-Sepharose column at 4° C. at a flow rate of 0.2 ml/min until the protein was bound to the resin. The column was washed with an excess of buffer B (KH₂PO₄10 mM pH 7, KC1 1M). Protein was eluted at 0.2 min/ml with an elution buffer (100 mM TES, 10 mM DTT, 2 mM EDTA, DHF 8.5 mM), the elution profile was collected in 1 ml fractions using a Pharmacia LKB Frac 200 collector. Finally, the protein was desalted using molecular size exclusion chromatography in a Pharmacia G-25 column. Fractions containing DHFR activity were pooled and concentrated by ultrafiltration using centripreps Amicon.

The purity of the protein preparation was assessed by detection of a single band of the correct molecular weight stained with coomasie blue following a 12% SDS-PAGE. Protein concentration was determined by the method of Bradford using a BSA as a standard. DHFR activity was measured spectrophotometrically by monitoring the decrease in absorbance at 340 mm. The assay reaction (1 ml) contained 50 mM TES, 1 mM EDTA, 75 mM 2-mercaptoethanol PH 7, 100 mM NADPH, 1 mg/ml BSA 30 mM DHF and 1-10 units of enzyme. One unit of enzyme, is defined as the amount of enzyme which produces 1 nmol of product per min using ε of 12300M⁻¹ at 340 nm.

Inhibition of dihydrofolate reductases by 1,2-dihydro-1,3,5 triazines was analysed with T. cruzi and human DHFR.

DHFR assays contained 30 mM dihydrofolate, 0.1 mM NADPH, 50 mM TES buffer pH 7, 75 mM 2-mercaptoethanol and specified amounts of inhibitors. The reaction was initiated by addition of 2.8 or 2.3 units of T. cruzi and human, respectively, and monitored at 340 nm. The Kₘ values used were 1.2 and 0.6 for T. cruzi and human DHFR, respectively and had been previously determined using different concentrations of DHF in a Lineweaver-Burk plot. The concentration of inhibitor which produced 50% of inhibition (I₅₀) in the conditions described, was determined by interpolation of plots of % inhibition against inhibitor concentrations. The concentrations used in the assay typically spanned between 10-80% inhibition.

Enzyme Assays and Inhibition by Cyc Analogues.

The activities of wild-type and A16V+S108T mutant pfDHFRs were determined spectrophotometrically according to the literature (Sirawaraporn, W.; Prapunwattana, P.; Sirawaraporn, R.; Yuthavong, Y.; Santi, D. V. The Dihydrofolate Reductase Domain of Plasmodium falciparum Thymidylate Synthase-Dihydrofolate Reductase. J.Biol. Chem. 1993, 29, 21637-44).

The reaction solution (1 mL) contained 1×DHFR buffer (50 mM TES, pH 7.0, 75 mM 2-mercaptoethanol, 1 mg/mL Bovine Serum Albumin), 100 mM each of the substrate H₂folate and cofactor NADPH, and appropriate amount (0.001-0.005 units) of the affinity-purified enzymes. Inhibition of the enzymes by Cyc and its analogues was carried out by determination of the Kᵢ values of the inhibitors for the enzymes by fitting to the equation IC₅₀=Kᵢ (1+([S]/Kₘ)), (Segal, I. H. in “Enzyme Kinetics: Behavior and Analysis of Steady-State and Rapid Equilibrium Enzyme Systems” (I. H. Segal, Ed.) pp. 100-160, Wiley-Interscience, New York) where IC₅₀ is the concentration of inhibitor which inhibits 50% of the enzyme activity under the standard assay condition and Kₘ is the Michaelis constant for the substrate H₂folate. The resistance factor which determines the effectiveness of the inhibitor against the mutant DHFR over the wild-type enzyme were assessed from the values of the ratios of the Kᵢ for the A16V+S108T mutant enzyme and the wild-type enzyme (Kᵢ-mut/Kᵢ-wt).

Plasmodium falciparum Culture and Drug Screening

Two clones of Plasmodium. falciparum, Tm4/8.2 (Wild type DHFR) and T9/94 (A16VS108T) [Thaithong, S.; Chan, S- W.; Songsomboon, S.; Wilairat, P.; Seesod, N.; Sueblinwong, T.; Goman, M.; Ridley, R.; Beale, G. Pyrimethamine resistant mutations in Plasmodium falciparum. Mol. Biochem. Parasitol. 1992, 52, 149-158] were maintained continuously in human erythrocytes at 37° C. under 3% CO₂ in RPMI 1640 culture media supplemented with 25 mM HEPES, pH 7.4, 0.2% NaHCO₃, 40 μg/ml gentamicin and 10% human serum [Trager, W.; and Jensen, J. B. Human malarial parasites in continuous culture. Science, 1976, 193, 673-675]. In vitro antimalarial activity was determined by using [³H]-hypoxanthine incorporation method [Desjardins, R. E.; Canfield, C. J.; Haynes, J. D.; Chulay, J. D. Quantitative assessment of antimalarial activity in vitro by a semiautomated microdilution technique. Antimicrob Agents Chemother. 1979, 16, 710-718]. The drugs were initially dissolved in DMSO and diluted with the culture media. Aliquots (25 μl) of drug solution of different concentrations was placed in 96-well plate together with 200 μl of 1.5% cell suspension of parasitized erythrocytes containing 1-2% parasitemia. Final concentration of DMSO was 0.1% where there was no effect on the parasite growth. The mixtures were incubated in a 3% CO₂ incubator at 37° C. After 24 h of incubation, 25 μl (0.25 μCi) of [³H]-hypoxanthine were added to each well. The mixtures were further incubated under the same condition for 18-24 h. DNA of parasites was harvested onto glass filter paper (Unifilter, Packard, USA). The filters were dried and liquid scintillation fluid was added for radioactivity measurement in a 6-probe liquid scintillation-counter (Packard, USA). A 50% inhibitory concentration (IC₅₀) value was determined from the sigmoid curve of percent [³H]-hypoxanthine incorporation against drug concentration.

Analysis of the Interaction of Human and T. cruzi DHFR with 1.2-dihydro-1,3,5-triazines

Selective inhibition of dihydrofolate reductase by 1,2-dihydro-1,3,5-triazines was analysed with T. cruzi and human DHFR. DHFR assays contained 30 μM dihydrofolate, 0.1 mM NADPH, 50 mM TES buffer pH 7, 75 mM β-mercaptoethanol and specified amounts of inhibitors. The reaction was initiated by addition of 2.8 or 2.3 units of T. cruzi and human enzyme, respectively, and monitored at 340 nm. The Km values used were 1.2 and 0.64 μM for T. cruzi and human DHFR, respectively and had been previously determined from Lineweaver-Burk plots using different concentrations of DHF. The concentration of inhibitor which produced 50% of inhibition (I50) in the conditions described, was determined by interpolation of plots of % inhibition against inhibitor concentrations. The concentrations used in the assay typically spanned between 10-80% inhibition.

KiI 50=[I]_{0.5}/((1+[S])/Km)

The results of the inhibitor binding studies are shown in tables 1 to 8.

TABLE 1
Inhibition constants (Kᵢ) of cycloguanil and some analogues of formula
(I) (Y = H, R¹ = R² = R = H) against the wild-type (wt) and
A16V.S108T mutant (mut) of DHFR from Plasmodium falciparum. IC₅₀ values
(nM) of the cycloguanil analogues with wild-type (TM4/8.2)
and resistant T9/94 strains of P. falciparum. The T9/94 resistant
strain harbours the A16V.S108T mutant of pfDHFR.

| | | | | | | Kᵢ(mut)/Kᵢ | | |
|---|---|---|---|---|---|---|---|---|
| R | X | Kᵢ(wt) nM | Rel. to Cyc. | Kᵢ(mut) nM | Rel. to Cyc. | (wt) | TM4/8.2 | T9/94 |
| H | Cl | 24.4 ± 4.3 | 16 | 649 ± 77 | 0.5 | 26 | 952 | 313 |
| H | H | 329 ± 27 | 220 | 585 ± 70 | 0.4 | 1.8 | | |
| H | F | 270 ± 28 | 180 | 469 ± 71 | 0.4 | 1.7 | | |

TABLE 2
Inhibition constants (Kᵢ) of cycloguanil analogues of formula (Ia)
(X = Cl, Y = H, R¹ = H) against the wild-type (wt) and A16V.S108T
mutant (mut) of DHFR from Plasmodium falciparum. IC₅₀ values (nM) of
the cycloguanil analogues with wild-type (TM4/8.2) and resistant
T9/94 strains of Plasmodium falciparum. The T9/94 resistant strain
harbours the A16V.S108T mutant of pfDHFR.

| R² | Kᵢ(wt) nM | Rel. to Cyc. | Kᵢ(mut) nM | Rel. to Cyc. | Kᵢ(mut)/Kᵢ(wt) | TM4/8.2 | T9/94 |
|---|---|---|---|---|---|---|---|
| phenyl | 4.5 ± 0.2 | 3.0 | 49.3 ± 3.3 | 0.038 | 11.0 | 27 | 439 |
| p-chlorophenyl | 7.5 ± 0.9 | 5.0 | 105 ± 17 | 0.1 | 14.0 | 499 | 386 |
| p-fluorophenyl | 8.6 ± 1.4 | 5.7 | 130 ± 17 | 0.1 | 15.1 | 359 | 395 |
| p-cyanophenyl | 11.3 ± 1.4 | 7.5 | 205 ± 22 | 0.2 | 18.2 | 804 | 323 |
| p-isopropylphenyl | 1.9 ± 0.6 | 1.3 | 15.2 ± 1.6 | 0.01 | 8.0 | 973 | 357 |
| 4-biphenyl | 1.3 ± 0.1 | 0.9 | 8.5 ± 1.0 | 0.006 | 6.5 | 621 | 365 |
| p-hydroxyphenyl | 11.3 ± 1.7 | 7.5 | 98 ± 10 | 0.074 | 8.6 | | |
| m-hydroxyphenyl | 6.0 ± 0.4 | 4.0 | 123 ± 13 | 0.094 | 20.6 | 48 | 280 |
| p-methoxyphenyl | 11.4 ± 1.5 | 7.6 | 129 ± 25 | 0.1 | 11.3 | 424 | 346 |
| m-methoxyphenyl | 10.4 ± 1.4 | 6.9 | 62.8 ± 2.4 | 0.05 | 6.0 | 577 | 318 |
| p-ethoxyphenyl | 1.5 ± 0.2 | 1.0 | 36 ± 6 | 0.028 | 24.3 | 181 | 145 |
| p-butyloxyphenyl | 1.0 ± 0.1 | 0.7 | 5.0 ± 0.6 | 0.0038 | 5.0 | 287 | 110 |
| p-benzyloxyphenyl | 0.6 ± 0.0 | 0.4 | 4.4 ± 0.8 | 0.0033 | 7.3 | 107 | 30 |
| m-benzyloxyphenyl | 0.7 ± 0.0 | 0.5 | 6.2 ± 0.5 | 0.005 | 8.8 | 832 | 406 |
| p-phenoxyphenyl | 0.4 ± 0.0 | 0.3 | 3.8 ± 0.4 | 0.003 | 9.5 | 121 | 35 |
| m-phenoxyphenyl | 0.5 ± 0.0 | 0.3 | 2.7 ± 0.3 | 0.002 | 5.4 | 367 | 341 |
| 3-(4-methoxyphenoxyl)phenyl | 0.7 ± 0.0 | 0.5 | 6.6 ± 0.5 | 0.005 | 9.4 | 2876 | 441 |
| 3-(4-chlorophenoxy)phenyl | 1.4 ± 0.3 | 0.9 | 7.5 ± 0.5 | 0.005 | 5.3 | 2853 | 428 |
| 3-(4-t-butylphenoxy)phenyl | 1.7 ± 0.0 | 1.1 | 9.9 ± 1.5 | 0.008 | 5.8 | 4118 | 1459 |
| 3,4-dimethoxyphenyl | 32.7 ± 2 | 22 | 124 ± 5 | 0.09 | 3.8 | 2269 | 352 |
| 3,5-dimethoxyphenyl | 38.3 ± 8 | 25 | 100 ± 7 | 0.08 | 2.6 | 3253 | 276 |

TABLE 3
Inhibition constants (Kᵢ) of cycloguanil analogues of formula (Ib)
(X = H, Y = Cl) against the wild-type (wt) and A16V.S108T mutant
(mut) of DHFR from Plasmodium falciparum. IC₅₀ values (nM) of
the cycloguanil analogues with wild-type (TM4/8.2) and resistant T9/94
strains of Plasmodium falciparum. The T9/94 resistant strain
harbours the A16V.S108T mutant of pfDHFR.

| R¹ | R² | Kᵢ(wt) nM | Rel. to Cyc. | Kᵢ(mut) nM | Rel. to Cyc. | Kᵢ(mut)/Kᵢ(wt) | TM4/8.2 | T9/94 |
|---|---|---|---|---|---|---|---|---|
| H | phenyl | 11.7 ± 2.5 | 7.8 | 10 ± 7 | 0.008 | 0.9 | 565 | 24 |
| H | 4-methoxyphenyl | 21.8 ± 1.0 | 14.5 | 12.5 ± 0.9 | 0.009 | 0.6 | 2558 | 32 |
| H | 3-methoxyphenyl | 5.4 ± 0.2 | 3.6 | 59.1 ± 6.3 | 0.04 | 10.9 | 4373 | 31 |
| H | 4-phenoxyphenyl | 0.7 ± 0.0 | 0.5 | 2.7 ± 0.4 | 0.002 | 3.9 | 647 | 4 |
| H | 3-phenoxyphenyl | 1.0 ± 0.2 | 0.7 | 1.9 ± 0.2 | 0.0014 | 1.9 | 2299 | 29 |
| H | 3-(4-chlorophenoxy)phenyl | 1.3 ± 0.0 | 0.9 | 3.5 ± 0.5 | 0.003 | 2.7 | 3949 | 378 |
| H | 3-(4-methoxyphenoxy)phenyl | 2.3 ± 0.1 | 1.5 | 2.5 ± 0.3 | 0.002 | 1.1 | 4121 | 372 |
| H | 3-(4-t-butylphenoxy)phenyl | 7.7 ± 0.5 | 5.1 | 5.6 ± 0.9 | 0.004 | 0.7 | 5036 | 2883 |
| H | 3,4-dimethoxyphenyl | 27.2 ± 2 | 18.1 | 164 ± 6 | 0.1 | 6.0 | 6816 | 141 |
| H | 3,5-dimethoxyphenyl | 79.4 ± 7 | 52.9 | 30.9 ± 3 | 0.02 | 0.4 | >50000 | 33 |
| H | 3,4,5-trimethoxyphenyl | 960 ± 79 | 653.5 | 1011 ± 27 | 0.8 | 1.0 | >50000 | 4335 |
| H | 3-benzyloxyphenyl | 2.3 ± 0.5 | 1.5 | 3.2 ± 0.2 | 0.002 | 1.4 | 3971 | 234 |
| H | 3-(3,5-dichlorophenoxy)phenyl | 1.8 ± 0.6 | 1.2 | 4.7 ± 0.4 | 0.003 | 2.6 | 5473 | 42 |
| H | 3-hydroxyphenyl | 27.9 ± 4.5 | 18.6 | 19.7 ± 2.4 | 0.01 | 0.7 | 551 | 37 |
| H | 4-(n-propyloxy)phenyl | 3.3 ± 0.4 | 2.2 | 9.3 ± 0.6 | 0.007 | 2.8 | 248 | 4 |
| H | 4-bromophenyl | 27.0 ± 4.5 | 18 | 21.9 ± 2.5 | 0.017 | 0.8 | 9104 | 28 |
| H | 3-(3,4-dichlorophenoxy)phenyl | 2.2 ± 0.2 | 1.5 | 9.1 ± 0.7 | 0.007 | 4.1 | 3896 | 204 |
| H | 4-(3,5-difluorobenzyloxy)phenyl | 4.4 ± 0.4 | 2.9 | 7.4 ± 0.7 | 0.006 | 1.7 | 3781 | 38 |
| H | 4-hydroxyphenyl | 59.1 ± 1.0 | 39.4 | 57.7 ± 7.5 | 0.044 | 1.0 | 3068 | 46 |

TABLE 4
Inhibition constants (Kᵢ) of cycloguanil analogues of formula (Ic)
(X = Y = Cl) against the wild-type (wt) and A16V.S108T mutant
(mut) of DHFR from Plasmodium falciparum. IC₅₀ values (nM) of
the cycloguanil analogues with wild-type (TM4/8.2) and resistant T9/94
strains of Plasmodium falciparum. The T9/94 resistant strain
harbours the A16V.S108T mutant of pfDHFR.

| R¹ | R² | Kᵢ(wt) nM | Rel. to Cyc. | Kᵢ(mut) nM | Rel. to Cyc. | Kᵢ(mut)/Kᵢ(wt) | TM4/8.2 | T9/94 |
|---|---|---|---|---|---|---|---|---|
| H | H | 5.3 ± 0.7 | 3.5 | 43.6 ± 5.8 | 0.033 | 8.2 | 123 | 32 |
| H | Ph | 1.6 ± 0.2 | 1.1 | 11.0 ± 1.8 | 0.008 | 6.9 | 26 | 29 |
| H | 3-phenoxyphenyl | 1.9 ± 0.3 | 1.3 | 5.2 ± 0.6 | 0.004 | 2.7 | 330 | 29 |
| H | 3-(4-methoxyphenoxy)phenyl | 1.9 ± 0.3 | 1.3 | 7.4 ± 0.8 | 0.006 | 3.9 | 2931 | 241 |

TABLE 5
Inhibition constants (Kᵢ) of cycloguanil analogues of formulae
(Id₁), (Id₂), (Id₃), and (I) against the wild-type (wt) and A16V.S108T
mutant (mut) of DHFR from Plasmodium falciparum. IC₅₀ values
(nM) of the cycloguanil analogues with wild-type (TM4/8.2) and resistant
T9/94 strains of Plasmodium falciparum. The T9/94 resistant
strain harbours the A16V.S108T mutant of pfDHFR.

| | | | | | | Rel. to | | | | | |
|---|---|---|---|---|---|---|---|---|---|---|---|
| | | | | | Kᵢ(wt) nM | Cyc. | Kᵢ(mut) nM | Rel. to Cyc. | Kᵢ(mut)/Kᵢ(wt) | TM4/8.2 | T9/94 |
| (Id₁) | R¹ | R² | | | | | | | | | |
| | H | 3-benzyloxyphenyl | | | 10.3 ± 2.6 | 6.9 | 11.7 ± 3.2 | 0.009 | 1.1 | 13981 | 1728 |
| (Id₂) | R¹ | R² | | | | | | | | | |
| | H | 3-phenoxyphenyl | | | 6.5 ± 0.5 | 4.3 | 10.8 ± 1.2 | 0.008 | 1.7 | 4108 | 4315 |
| | H | 3-(4-chlorophenoxy)phenyl | | | 219 ± 11 | 146 | 210 ± 18 | 0.2 | 0.9 | >10000 | >10000 |
| | H | 3-propyloxyphenyl | | | 1.3 ± 0.3 | 0.9 | 19.2 ± 2.7 | 0.015 | 14.8 | 25384 | 5410 |
| (Id₃) | H | 4-n-PrOC₆H₄ | | | 1.7 ± 0.3 | 1.1 | 6.1 ± 0.7 | 0.005 | 3.6 | 948 | 29 |
| | H | 4-n-BuOC₆H₄ | | | 3.2 ± 0.4 | 2.1 | 8.2 ± 0.8 | 0.006 | 2.6 | 1486 | 41 |
| | R¹ | R² | X | Y | | | | | | | |
| | H | 3-BnOC₆H₄ | H | BnO | 4.3 ± 0.2 | 2.9 | 7.2 ± 1.0 | 0.005 | 1.7 | | |
| | H | 3-BnOC₆H₄ | H | Bn | 2.3 ± 0.3 | 1.5 | 7.4 ± 0.9 | 0.006 | 3.2 | | |
| | H | 3-BnOC₆H₄ | BnO | H | 1.3 ± 0.2 | 0.9 | 5.7 ± 0.6 | 0.004 | 4.4 | 3447 | 255 |
| | H | 3-BnOC₆H₄ | H | Cl | 2.3 ± 0.5 | 1.5 | 3.2 ± 0.3 | 0.002 | 1.4 | 3971 | 234 |
| | H | 4-n-PrOC₆H₄ | H | Bn | 1.0 ± 0.2 | 0.7 | 4.8 ± 0.4 | 0.004 | 4.8 | 438 | 6 |
| | H | 4-n-BuOC₆H₄ | H | Bn | 2.1 ± 0.2 | 1.4 | 5.4 ± 0.5 | 0.004 | 2.6 | 1093 | 35 |
| | H | 3-PhOC₆H₄ | H | Bn | 2.9 ± 0.4 | 1.9 | 6.7 ± 0.8 | 0.005 | 2.3 | 934 | 30 |
| | H | H | H | Bn | 9.4 ± 0.4 | 6.3 | 6.0 ± 0.1 | 0.005 | 0.6 | 403 | 51 |
| | H | 3-PhOC₆H₄ | H | Ph | 2.0 ± 0.0 | 1.5 | 4.8 ± 0.9 | 0.004 | 2.2 | | |
| | H | H | 4-BrC₆H₄ | H | 11.3 ± 0.8 | 7.5 | 542 ± 35 | 0.413 | 48 | 727 | 346 |
| | H | H | BnO | H | 22.4 ± 1.3 | 14.9 | 28.6 ± 2.2 | 0.022 | 1.3 | 690 | 34 |
| | H | 4-n-BuOC₆H₄ | BnO | H | 0.8 ± 0.1 | 0.5 | 6.6 ± 0.6 | 0.005 | 8.2 | 384 | 4 |
| | H | 3-BnOC₆H₄ | BnO | H | 1.3 ± 0.2 | 0.9 | 5.7 ± 0.6 | 0.004 | 4.4 | 3447 | 255 |
| | H | 3-PhOC₆H₄ | BnO | H | 0.9 ± 0.2 | 0.6 | 4.5 ± 0.6 | 0.003 | 5 | 255 | 1678 |

TABLE 5a
In vitro inhibition (ED50 in μg/ml) of P. falciparum 3D7
and FCR3 strains and toxicity (ED50 in μg/ml) by cycloguanil analogues (I).
Also in vivo activity against Plasmodium berghei in mice.

| | | | | | | | | in vivo data | | |
|---|---|---|---|---|---|---|---|---|---|---|
| | | | | | | | Dose | Schedule | Mean % infected | |
| R¹ | R² | X | Y | Pf-3D7 | Pf-FCR3 | Toxicity | mg/kg | daily | RBC | SD |
| H | 4-PhC₆H₄ | Cl | H | 2.2 | 0.68 | 8.4 | | | | |
| H | 4-nBuOC₆H₄ | Cl | H | 0.14 | 0.091 | 50.0 | | | | |
| H | 3-BnOC₆H₄ | Cl | H | 0.18 | 0.62 | 7.1 | | | | |
| H | 4-PhOC₆H₄ | Cl | H | 0.07 | 0.2 | 33.8 | | | | |
| H | 4-BnOC₆H₄ | Cl | H | 0.26 | — | 106 | | | | |
| H | 3-PhOC₆H₄ | Cl | H | 0.28 | 0.32 | 56.0 | | | | |
| H | 3-(4-MeOPhO)C₆H₄ | Cl | H | 5.19 | 1.25 | 134 | | | | |
| H | 3-(4-tBuPhO)C₆H₄ | Cl | H | 0.75 | 0.26 | 12.1 | | | | |
| H | H | Cl | H | 0.04 | 0.13 | | | | | |
| H | 3-PhOC₆H₄ | H | Cl | 0.22 | 0.0008 | 10.6 | 25 | x4 | 7.05 | 1.43 |
| H | 3-(4-tBuPhO)C₆H₄ | H | Cl | >0.3 | 0.47 | 1.4 | | | | |
| H | 4-PhOC₆H₄ | H | Cl | — | 0.004 | 1.2 | 25 | b | 11.42 | 1.07 |
| H | 3-(4-ClPhO)C₆H₄ | H | Cl | 0.36 | 0.078 | | 25 | x4 | 5.0 | 3.00 |
| H | 3-BnOC₆H₄ | H | Cl | 0.45 | 0.019 | | | | | |
| H | 4-nBuOC₆H₄ | H | Cl | 0.3 | 0.0038 | 6.8 | 25 | x4 | 16.46 | 2.03 |
| H | 3-(3,4-Cl₂PhO)C₆H₄ | H | Cl | | | | 25 | x4 | 13.0 | 2.11 |
| H | 3-(4-MeOPhO)C₆H₄ | H | Cl | | | | 25 | x4 | 1.94 | 0.91 |
| H | 4-(4-CF₃PhO)C₆H₄ | H | Cl | | | | 24 | x4 | 4.4 | 0.35 |
| Chloroquine | | | | | | | 10 | x4 | 3.58 | 1.21 |
| 0.54 | | | | | | | | | | |
| Control | | | | | | | — | — | 22.94 | 3.34 |
| 1.49 | | | | | | | | | | |

TABLE 6
Inhibition constants Kᵢ (I₅₀) for cycloguanil
analogues (Ia) (X = Cl, Y = H) with DHFR-TS from
Trypanosoma cruzi. and human DHFR. For cycloguanil
Kᵢ = 370 nM (T. cruzi); Kᵢ = 16 nM
(human); selectivity = 0.043.

| | T. cruzi | Human | |
|---|---|---|---|
| R² | Kᵢ (I₅₀)(nM) | Kᵢ (I₅₀)(nM) | Selectivity |
| H | 16 | 1212 | 76 |
| p-fluorophenyl | 1010 | 10550 | 10 |
| p-cyanophenyl | 650 | 8080 | 12 |
| p-isopropylphenyl | 60 | 10050 | 167 |
| p-ethoxyphenyl | 70 | 1730 | 25 |
| p-methoxyphenyl | 26 | 5530 | 212 |
| phenyl | 42 | 3870 | 92 |
| m-phenoxyphenyl | 21 | 190 | 9 |
| p-n-butoxyphenyl | 26 | 400 | 15 |
| p-chlorophenyl | 270 | 2960 | 11 |
| p-phenoxyphenyl | 3 | 1129 | 376 |
| 3,5-dimethoxyphenyl | 200 | 25760 | 129 |
| 3,4-dimethoxyphenyl | 386 | 25680 | 67 |
| 3(4-methoxyphenoxy)phenyl | 48 | 830 | 17 |
| 3(4-t-butyl-phenoxy)phenyl | 112 | 770 | 7 |
| 4-benzyloxyphenyl | 13 | 1110 | 85 |

TABLE 7
Inhibition constants Kᵢ for cycloguanil analogues for
formula (I) with DHFR-TS from Trypanosoma cruzi and human DHFR.
For cycloguanil Kᵢ = 370 nM (T. cruzi);
Kᵢ = 16 nM (human); selectivity = 0.043

| | | | | T.cruzi | Human | |
|---|---|---|---|---|---|---|
| R¹ | R² | X | Y | Kᵢ(I₅₀)(nM) | Kᵢ(I₅₀)(nM) | Selectivity |
| H | 3-BnOC₆H₄ | H | CH₂O-2,3,5-C₆H₂Cl₃ | 30 | 22 | 0.73 |
| H | 3-BnOC₆H₄ | H | BnO | 128 | 2130 | 16.6 |
| H | 3-BnOC₆H₄ | H | Bn | 32 | 292 | 9.1 |
| H | 3-BnOC₆H₄ | BnO | H | 40 | 319 | 8.0 |
| H | 4-nBuOC₆H₄ | BnO | H | 49 | 203 | 4.1 |
| H | 3-PhOC₆H₄ | H | Cl | 4 | 2 | 0.5 |
| H | 3-PhOC₆H₄ | BnO | H | 3 | 10 | 3.3 |
| H | 4-MeOC₆H₄ | F | H | 61 | 26900 | 441 |
| H | 3-BnOC₆H₄ | H | Cl | 12 | 46 | 3.8 |
| H | 3-MeOC₆H₄ | H | Cl | 240 | 1270 | 5.3 |
| H | H | BnO | H | 10 | 44 | 4.4 |
| H | H | H | Bn | 29 | 66 | 2.3 |
| H | H | Br | H | 100 | 480 | 4.8 |
| H | H | Cl | Cl | 36 | 70 | 1.9 |

TABLE 8
Inhibition constants Kᵢ for cycloguanil analogues for
formula (I) with human DHFR and comparison with that of T. cruzi and P.
falciparum wild type (wt) and A16V.S108V mutant (mut).

| | | | | Kᵢ (human)/Kᵢ | Kᵢ (human)/Kᵢ | Kᵢ (human)/Kᵢ |
|---|---|---|---|---|---|---|
| R² | X | Y | Kᵢ (nM) | (T. cruzi) | (P. falc. wt) | (P. falc. mut) |
| H | Cl | H | 1212 | 75.7 | 49.7 | 1.9 |
| 3-HOC₆H₄ | Cl | H | 1680 | 56 | 280 | 13.7 |
| 4-MeOC₆H₄ | Cl | H | 5530 | 212 | 485 | 42.9 |
| Ph | Cl | H | 3870 | 92.1 | 860 | 78.5 |
| 4-iPrC₆H₄ | Cl | H | 10,050 | 167 | 5289 | 661 |
| 4-EtOC₆H₄ | Cl | H | 1730 | 24.7 | 1153 | 48 |
| 4-nBuOC₆H₄ | Cl | H | 400 | 15.3 | 400 | 80 |
| 4-ClC₆H₄ | Cl | H | 2960 | 11.0 | 395 | 28 |
| 4-FC₆H₄ | Cl | H | 10550 | 10.4 | 1227 | 81 |
| H | H | H | 1430 | — | 4.35 | 2.44 |
| H | F | H | 1800 | — | 6.67 | 3.84 |
| 4-MeOC₆H₄ | F | H | 26900 | 441 | — | — |
| H | Cl | Cl | 7 | 0.19 | — | — |
| H | BnO | H | 44 | 4.44 | 1.96 | 1.54 |
| H | H | Bn | 66 | 2.27 | 7.0 | 11 |
| 4-HOC₆H₄ | H | Cl | 56 | 1.17 | 0.95 | 0.97 |
| 3-PhOC₆H₄ | H | Cl | 2 | 0.5 | 1.8 | 0.8 |
| 3-BnOC₆H₄ | H | CH₂O-2,4,5-C₆H₄Cl₃ | 22 | 0.7 | 2.1 | 1.9 |
| 3-PhOC₆H₄ | BnO | H | 10 | 3.33 | 11 | 2.2 |
| 4-nBuOC₆H₄ | BnO | H | 203 | 4.1 | 253 | 31 |
| 3-BnOC₆H₄ | BnO | H | 319 | 8.0 | 245 | 56 |
| 3-BnOC₆H₄ | H | Bn | 292 | 9.1 | 127 | 39 |
| Ph | Cl | Cl | 280 | — | 175 | 25 |

The ratio of the inhibition constants for cycloguanil (A) with the wild-type (wt) (1.5 nM) and A16V.S108T mutant DHFR (1313 nM) is 876. It is this resistance factor which must be reduced whilst maintaining the activity against the wt enzyme. By removing the gem-dimethyl group as in the compound where R¹═R²═H, X═Cl and Y═H this ratio drops to 26 (Table 1). This can be seen to arise from a combination of poorer binding to the wild-type enzyme (by factor of about 16) and enhanced binding to the mutant enzyme (by about 0.5). The marked improvement in the Ki(mut)/Ki(wt) ratio when the methyl groups are replaced by hydrogen and the p-Cl-substituents replaced by H or F suggests that the two binding sites are acting cooperatively but the loss of binding to the wild-type enzyme needs to be addressed.

The data in Table 2 show the effect of replacing one of the methyl groups of cycloguanil with H and the other with an aryl substituent. It is noteworthy that the inhibition constants for all of the compounds of formula (1a) are better than the compound where both substituents at C-2 are H (Table 1; R₁═R₂═H, X═Cl) for wt and mutant DHFR except for the last two entries which are both disubstituted phenyl groups. There are eight compounds of formula (1a) [R²=4-biphenyl, p-butyloxyphenyl,-p-benzyloxyphenyl, m-benzyloxyphenyl, p-phenoxyphenyl, m-phenoxyphenyl, 3-(4-methoxyphenoxy)phenyl and 3-(4-chlorophenoxy)phenyl] that have Ki(wt) values below cycloguanil (1.5 nM) against the wild-type enzyme. Significantly these same compounds are the best inhibitors of the mutant enzyme with Ki values of 8.5, 5.0, 4.4, 6.2, 3.8, 2.7, 6.6 and 7.5 nM respectively, giving Ki(mut)/Ki(wt) ratios of 6.5, 5.0, 7.3, 8.8, 9.5, 5.4, 9.4 and 5.3 respectively. Clearly replacing the gem-dimethyl group with the m- or p-alkoxy-, aryloxy- or -arylalkoxy-phenyl group and H has improved the activity against the wt enzyme and has almost restored the inhibition constant with the mutant enzyme (2.7 nM for R=m-phenoxyphenyl) to that of cycloguanil with the wild-type enzyme (1.5 nM). Thus activity against the A16V.S108T mutant can be restored to a similar value as cycloguanil against the wt.enzyme by an appropriate choice of C-2-substitutent.

When this group of compounds was investigated in vitro against the wild-type TM4/8.2 and T9/94 resistant strain of Plasmodium falciparum (which harbours the A16V.S108T mutant DHFR) only three compounds possessed IC50 values lower than 40 nM which is the IC50 value of cycloguanil against the wild-type TM4/8.2 strain of P.falciparum (Table 1). They are the compounds (I, X═Cl, Y═H) where R² is phenyl (39 nM), p-benzyloxyphenyl (30 nM) and p-phenoxyphenyl (35 nM). The IC50 value against the wild-type TM4/8.2 is only maintained when R² is phenyl.

Having established substituents at C-2 of cycloguanil (A) for the effective inhibition of the A16V.S108T pfDHFR, attention was turned to the substituents on the aromatic ring at N-1. It was known that in pyrimethamine (B), moving the p-chloro-substituents into the m-position was advantageous against the C59N.S108N mutant pfDHFR. The data in Table 3 show that moving the chloro-substituents from the p- to the m-position in the cycloguanil analogue (Compounds of formula (Ib)) decreases the activity against the wt enzyme but increases the activity against the mutant enzyme. In the compound of formula (Ib) (R²=Ph) the same is observed (cf., (Ia), R²=Ph, Table 2). However, when the phenyl ring is substituted with the 4-phenoxy-, 3-phenoxy- or -3-(4-chlorophenoxy)- not only does the inhibition constant become lower than that of cycloguanil against the wt enzyme but the analogue with the 3-phenoxyphenyl group has an inhibition of 1.9 nM against the A16V.S108T mutant DHFR, virtually identical, within the experimental error, with that of cycloguanil for the wt enzyme.

Nine compounds in this group of cycloguanil analogues have IC50 values below 40 nM against the resistant P. falciparum T9/94 strain. Two, namely (I, X═H, Y═Cl) R²=4-phenoxyphenyl and 4-(n-propyloxy)phenyl having exceptionally low IC50 values of 4 nM against the resistant strain T9/94 of P. falciparum. The activity against the wild-type strain however is not maintained.

Table 4 shows that for the inhibition of the wt enzyme and A16V.S108T pfDHFR by compounds of formula (Ic) compounds where R² is substituted phenyl, in particular 3-phenoxyphenyl or 3-(4-methoxyphenoxy)phenyl have good activity against the wt enzyme and very good activity against the mutant compared with cycloguanil.

Three of the four compounds in this group of cycloguanil analogues have IC50 values below 40 nM (i.e. 29-32 nM) against the resistant P. falciparum T9/94 strain. One, namely, (I, X═Y═Cl, R²=phenyl) maintains similar activity against the wild-type P. falciparum.

In order to investigate the effect of rigidifying the N-1 side chain of WR99210 cycloguanil analogues Id₁, and Id₂ were prepared.The data in Table 5 show that the partially rigidified N-1 side chain in compounds of formula (Id₁)

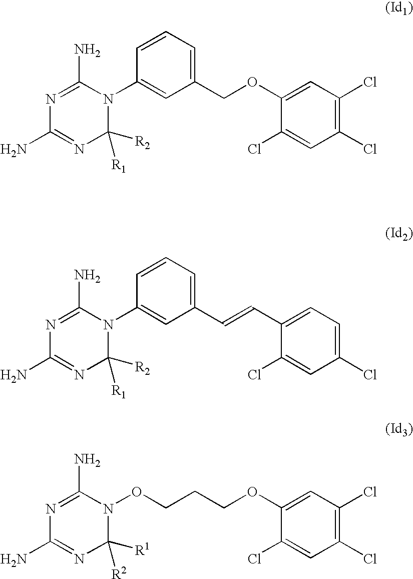

is very effective. However, this is not further enhanced by replacing the dimethyl group at C-2 with H and the 3 benzyloxyphenyl substituent (Compare Table 2 and 3 where these are amongst the best substituents at the C-2 when the substituent at N-1 is 4-chlorophenyl or 3-chlorophenyl). This is presumably due to interference between the N-1 and C-2 substituents in binding to the wt or mutant enzyme. Further rigidification in the analogues of formula (Id₂) leads to further loss of activity.

The present invention also provides compounds of formula (Id₃) wherein R¹ and R² are as defined above without any proviso together with pharmaceutical compositions containing them; they have the same uses as the compounds of formula (I) and (II). It is of interest to note that the flexible side chain of WR99210 in two (Id₃) compound in Table 5 is slightly less effective than 1-m-benzylphenyl-2-p-n-propoxyphenyl-4,6-diamino-1,2-dihydro-1,3,5-triazine, 1-m-benzylphenyl-2-p-n-butoxyphenyl-4,6-diamino-1,2-dihydro-1,3,5-triazine, 1-p-benzyloxyphenyl-2-p-n-butoxyphenyl-4,6-diamino-1,2-dihydro-1,3,5-triazine which have the same substituents at C-2. Thus the rather elaborate side chain of WR99210 is not necessary to achieve an effective inhibition of the mutant enzyme.

The next group of compounds in Table 5 (Compounds of formula (I) with R²=3-benzyloxyphenyl) have one of the best combinations of substituents at C-2 with variation of the substituent at N-1. It is of interest to note that of these compounds the one with Y=chlorine emerges as the best candidate for the mutant enzyme and also has an excellent Ki(mut)/Ki(wt) ratio of 1.4. However, although it is approaching the Ki(mut) values of the best compound of formula (Ib) R²=3-phenoxyphenyl, Table 3 it does not surpass it.

The next group of compounds in Table 5 have the 3-benzylphenyl substituent at N-1 and as can be seen all four compounds investigated have Ki values for the wt and mutant enzymes below nM, the most effective inhibitor against both the wt and mutant enzymes being 1-m-benzylphenyl-2-p-n-propoxyphenyl-4,6-diamino-1,2-dihydro-1,3,5-triazine. The next group of compounds shows again several very promising inhibitors against both wt and mutant enzymes. These include 1-m-biphenyl-2-m-phenoxyphenyl-4,6-diamino-1,2-dihydro-1,3,5-triazine, 1-p-benzyloxyphenyl-2-p-n-butoxyphenyl4,6-diamino-1,2-dihydro-1,3,5-triazine, 1-p-benzyloxyphenyl-2-m-benzyloxyphenyl-4,6-diamino-1,2-dihydro-1,3,5-triazine, 1-p-benzyloxyphenyl-2-m-phenoxyphenyl-4,6-diamino-1,2-dihydro-1,3,5-triazine.

Although six compounds in this group showed IC50 values below 40 nM against the resistant P. falciparum T9/94 strain, two, namely (I, X═H, Y=benzyl, R²=3-benzyloxyphenyl and I, X=benzyloxy, Y═H, R²=4-n-butoxyphenyl) showed exceptional activity with IC50 values of 6 and 4 nM respectively.

Some of the cycloguanil analogues (I) have been investigated in vitro against two different strains of of P. falciparum (Table 5a). Pf-3D7 is a wild-type strain and Pf-FCR3 is a resistant strain containing the A16V.S108T DHFR mutant. Three analogues (I, X═H, Y═Cl, R²=3-phenoxyphenyl, 4-phenoxyphenyl and 4-n-butoxyphenyl all showed ED50 values in the nanomolar range. The most active analogue also had the lowest toxicity. These analogues were tested in vivo in a mice against Plasmodium berghei. It is important to appreciate that this is the wild-type parasite whereas the analogues were selected for their ability to inhibit the A16V.S108T mutant pfDHFR. It is likely therefore that against the resistant strain they would be more effective in vivo. Nevertheless the analogue (I, X═H, Y═Cl, R²=3-phenoxyphenyl) which has an ED50=0.8 nM against the resistant Pf FCR-3 strain in vitro was also the most effective analogue in vivo.

Cycloguanil is a much poorer inhibitor of tcDHFR than pf DHFR with Kᵢ=370 nM. The data in Table 6 show that by removing both C-2 methyl groups of the cycloguanil, a compound (of formula (Ia) R²═H) with increased activity is obtained suggesting that there is some steric hindrance in the tcDHFR active site at this position. Moreover, introducing any substituent at C-2 appears to decrease the binding capacity of the inhibitor with the exception of 1-p-chlorophenyl-2-p-phenoxyphenyl-4,6-diamino-1,2-dihydro-1,3,5-triazine which becomes a lead compound for this parasite. Indeed all the Kᵢ values (for compounds of formula (Ia), where R²=aryl) are higher than those for the same compounds against the A16V.S108T resistant strain of P.falciparum. This suggests that there may be greater steric restriction at this position in the T.cruzi enzyme than in the A16V.S108T mutant pfDHFR, although clearly the enzyme is sensitive to the structure of the aryl group and with m-phenoxyphenyl and 4-phenoxyphenyl the steric factor is largely offset by additional binding.

Before undertaking in vitro and in vivo evaluation of the best inhibitors of A16V.S108T pfDHFR and tc DHFR their inhibition constant against human DHFR was investigated. The data is shown in Tables 6-8. Since it is desirable to have a good selectivity index these data help to eliminate what would otherwise be regarded as good candidates to go forward. If both the selectivity index and the absolute Kᵢ(IC₅₀) value are taken into consideration then for Trypanosoma cruzi 1-p-chlorophenyl-2-(p-phenoxyphenyl)-4,6-diamino-1,2-dihydro-1,3,5-triazine [Kᵢ(IC₅₀)=3 nM, selectivity=376] is the compound of choice (Table 6). However, other candidates are not discarded since there are other factors involved in the in vivo effectiveness. Thus, 1-p-benzyloxyphenyl-2-p-n-butyloxyphenyl-4,6-diamino-1,2-dihydro-1,3,5-triazine, 1-p-benzyloxyphenyl-2-m-benzyloxyphenyl-4,6-diamino-1,2-dihydro-1,3,5-triazine and 1-m-benzylphenyl-2-m-benzyloxyphenyl-4,6-diamino-1,2-dihydro-1,3,5-triazine could well have acceptable selectivity indices against both wt and resistant plasmodium falciparum.

It is noteworthy that the inhibitory activity of 1-m-chlorophenyl-2-m-phenoxyphenyl-4,6-diamino-1,2-dihydro-1,3,5-triazine, 1-[3-(2,4,5-trichlorophenoxymethyl)phenyl]-2,2-dimethyl4,6-diamino-1,2-dihydro-1,3,5-triazine, 1-m-chlorophenyl-2,2-dimethyl-4,6-diamino-1,2-dihydro-1,3,5-triazine and 1-p-benzyloxyphenyl-2-m-phenoxyphenyl-4,6-diamino-1,2-dihydro-1,3,5-triazine against human DHFR is comparable to that of methotrexate. Consequently these were examined as antiumour agents.

1. A compound of formula (I) or (II) 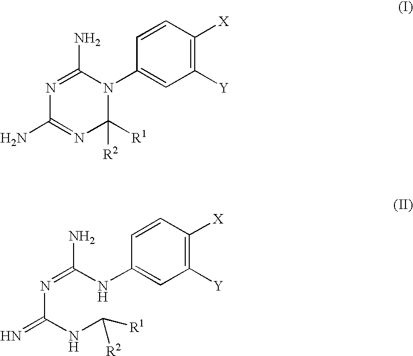
wherein X is hydrogen, halogen, alkyl, aralkyl, aryloxy, arylalkoxy or alkoxy,
Y is hydrogen, halogen, alkyl, aralkyl, aryloxy, arylalkoxy or alkoxy, 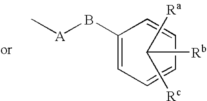
wherein A and B are different and one of A and B is CH₂ and the other is O, NH, or S or A and B are both CH₂ or CH═, and R^{a}, R^{b}, and R^{c} are the same or different and are hydrogen, halogen, alkyl, alkoxy, aryloxy, aralkyl or arylalkoxy
R¹ is hydrogen, and
R² is hydrogen, C₁-C₆ alkyl or aryl which is unsubstituted or substituted by halogen, cyano, hydroxy, C₁-C₆ alkyl
which is unsubstituted or substituted by halogen, cyano, hydroxy, C₁-C₆ alkoxy, aralkyl, aryloxy or aryl,
C₁-C₆ alkoxy, aralkyl, arylalkoxy, aryloxy or aryl
which is unsubstituted or substituted by halogen, cyano, hydroxy, C₁-C₆ alkyl or C₁-C₆ alkoxy; and pharmaceutically acceptable salts thereof; with the proviso that when R² is phenyl and Y is hydrogen, X is not chlorine.

2. A compound according to claim 1 wherein X is chlorine or hydrogen and Y is hydrogen or chlorine.

3. A compound according to claim 1 or 2 wherein R² is not hydrogen.

4. A compound according to any one of the preceding claims wherein R² is substituted phenyl wherein the substituents are alkoxy, aryloxy or aryl.

5. A compound according to any one of the preceding claims wherein Y is

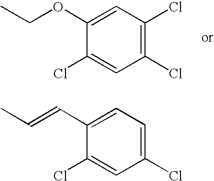

6. A compound according to claim 1 wherein X is chorine, Y is hydrogen, and R² is phenyl which is unsubstituted or substituted by alkyl, alkoxy, benzyloxy or phenyl which is unsubstituted or substituted by halogen, C₁-C₆ alkyl or C₁-C₆ alkoxy.

7. A compound according to claim 2 wherein R² is 4-biphenyl, 4-ethoxyphenyl, 4-butyloxyphenyl, 4-benzyloxyphenyl, 3-benzyloxyphenyl, 4-phenoxyphenyl, 3-phenoxyphenyl, 3-(4-methoxyphenoxy)phenyl, 3-(4-chlorophenoxy)phenyl or 3-(4-t-butylphenoxy)phenyl.

8. A compound according to claim 1 wherein X is hydrogen, Y is chlorine, and R² is phenyl substituted by unsubstituted or substituted phenoxy or benzyloxy.

9. A compound according to claim 4 wherein R² is 4-phenoxyphenyl, 3-phenoxyphenyl, 3-(4-chlorophenoxy)phenyl, 3-(4-methoxyphenoxy)phenyl, 3-(4-t-butylphenoxy)phenyl, 3-benzyloxyphenyl or 3-(3,5-dichlorophenoxy)phenyl.

10. A compound according to claim 1 wherein X and Y are both chlorine, and R² is substituted phenyl.

11. A compound according to claim 1 which is 1-p-chlorophenyl-4,6-diamine-1,2-dihydro-1,3,5-triazine, 1-p-chlorophenyl-2-p-isopropylphenyl-4,6-diamino-1,2-dihydro-1,3,5-triazine, 1-m-chlorophenyl-2-m-phenoxyphenyl-4,6-diamino-1,2-dihydro-1,3,5-triazine, 1-[3-(2,4,5-trichlorophenoxymethyl)phenyl]-2,2-dimethyl-4,6-diamino-1,2-dihydro-1,3,5-triazine, 1-p-benzyloxyphenyl-2-m-phenoxyphenyl-4,6-diamino-1,2-dihydro-1,3,5-triazine, 1-p-benzyloxyphenyl-2-p-n-butoxyphenyl-4,6-diamino-1,2-dihydro-1,3,5-triazine, 1-p-benzyloxyphenyl-2-m-benzyloxyphenyl-4,6-diamino-1,2-dihydro-1,3,5-triazine, 1-m-benzylphenyl-2-m-benzyloxyphenyl-4,6-diamino-1,2-dihydro-1,3,5-triazine.

12. A process for the production of a compound of formula (III) 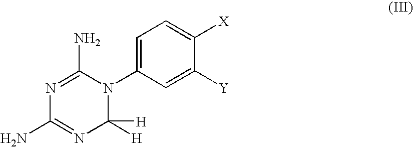
which process comprises a reacting compound of formula (IV) 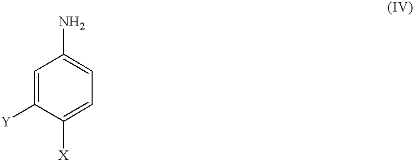
with dicyandiamide and dimethoxymethane, wherein X and Y are as defined in claim 1.

13. A process for the production of compounds of formula (V) 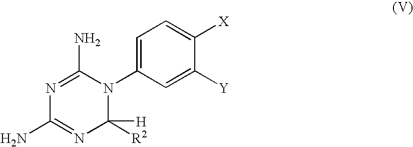
which process comprises reacting a compound of formula (VI) 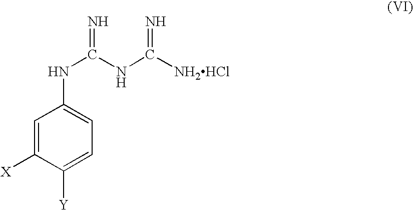
with a compound of formula (VII) or a ketal of (VII) 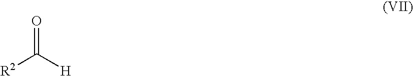
in the presence of a strong acid, wherein X and Y are as defined in claim 1 and R² is C₁-C₆ alkyl or aryl which is unsubstituted or substituted by halogen, cyano, hydroxy, C₁-C₆ alkyl
which is unsubstituted or substituted by halogen, cyano, hydroxy,
C₁-C₆ alkoxy, aralkyl, aryloxy or aryl,
C₁-C₆ alkoxy, aralkyl, arylalkoxy, aryloxy or aryl
which is unsubstituted or substituted by halogen, cyano, hydroxy, C₁-C₆ alkyl or C₁-C₆ alkoxy; with the proviso that when R₂ is phenyl and Y is hydrogen, X is not chlorine.

14. A pharmaceutical composition which comprises a compound of formula (I) or (II) 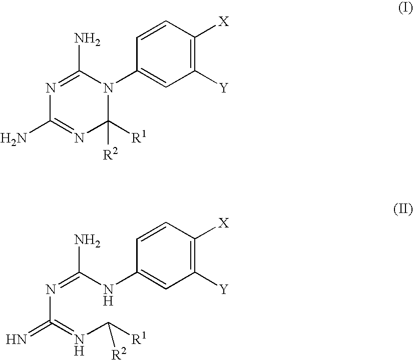
wherein X is hydrogen, halogen, alkyl, aralkyl, aryloxy, arylalkoxy or alkoxy,
Y is hydrogen, halogen, alkyl, aralkyl, aryloxy, arylalkoxy or alkoxy, 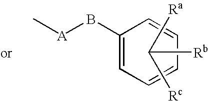
wherein A and B are different and one of A and B is CH₂ and the other is O, NH, or S or A and B are both CH₂ or CH═, and R^{a}, R^{b}, and R^{c} are the same or different and are hydrogen, halogen, alkyl, alkoxy, aryloxy, aralkyl or arylalkoxy.
R¹ is hydrogen, and
R² is hydrogen C₁-C₆ alkyl or aryl which is unsubstituted or substituted by halogen, cyano, hydroxy, C₁-C₆ alkyl
which is unsubstituted or substituted by halogen, cyano, hydroxy,
C₁-C₆ alkoxy, aralkyl, aryloxy or aryl,
C₁-C₆ alkoxy, aralkyl, arylalkoxy, aryloxy or aryl
which is unsubstituted or substituted by halogen, cyano, hydroxy, C₁-C₆ alkyl or C₁-C₆ alkoxy; and pharmaceutically acceptable salts thereof; together with a pharmaceutically acceptable carrier or diluent.

15. A compound as defined in claim 14 for use in the treatment or prophylaxis of malaria.

16. A compound as defined in claim 14 for use in the treatment or prophylaxis of drug resistant malaria.

17. A compound as defined in claim 14 for use in the treatment or prophylaxis of a strain of malaria which is resistant to cycloguanil.

18. A compound as defined in claim 14 for use in the treatment or prophylaxis of a disease caused by a parasite.

19. A compound as defined in claim 14 for use in the treatment or prophylaxis of Chagas' disease.

20. A compound as defined in claim 14 for use in the treatment or prophylaxis of sleeping sickness.

21. A compound as defined in claim 14 for use in the treatment or prophylaxis of Toxoplasma gondii.

23. Use of a compound as defined in claim 14 in the manufacture of a medicament for the treatment or prophylaris of malaria, a disease caused by a parasite, Chagas' disease, sleeping sickness or Toxoplasma goudii.
